(19) United States
(12) Patent Application Publication
(10) Pub. No.: US 2025/0164397 A1
(43) Pub. Date: May 22, 2025

(54) SULPHIDE SENSOR

(71) Applicant: Oxford University Innovation Limited, Oxford (GB)

(72) Inventors: Paul BEER, Oxford (GB); Edward MITCHELL, Oxford (GB)

(21) Appl. No.: 18/292,580

(22) PCT Filed: Jul. 27, 2022
(86) PCT No.: PCT/GB2022/051972

§ 371 Date: Jan. 26, 2024

(30) Foreign Application Priority Data
Aug. 5, 2021 (GB) 2111340.2

(51) Int. Cl. G01N 21/64; C07D 405/14; C07F 5/02; G01N 33/18

(52) U.S. Cl. CPC G01N 21/6428; C07D 405/14; C07F 5/022; G01N 33/182; G01N 2021/6439

(57) ABSTRACT

Compounds I suitable for use in sensing anions, such as sulphide are described. Also described is a method of detecting sulphide using the described compounds.

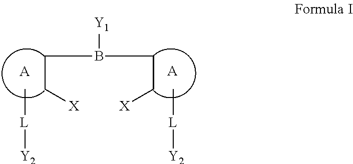

### INTRODUCTION

The present invention relates to new compounds, more specifically, those that are suitable for use in sensing sulphide anions. Even more specifically, the present invention relates to fluorophore-containing compounds that are suitable for this use. The present invention also relates to methods of detecting sulphide using the compounds of the present invention.

### BACKGROUND OF THE INVENTION

The critical roles of anions in a plethora of biological, medicinal and anthropogenic environmental processes has been crucial in the development of the burgeoning field of anion supramolecular chemistry.¹ One such example of this is the hydrosulfide anion (HS⁻). As is well known, hydrogen sulphide (H₂S) is a particularly toxic gas which can have a detrimental effect towards human health. Furthermore, this simple molecule has recently been recognised as a third gasotransmitter, along with carbon monoxide and nitric oxide, playing crucial signalling physiological functions in the regulation of cardiovascular, immune, endocrine and nervous systems.²⁻⁵ In addition, abnormal H₂S concentrations have been implicated in a number of medical conditions.^{6,7} With a pKₐ of 7.0, at physiological pH, the hydrosulfide anion (HS⁻) is the dominant species of H₂S in aqueous solution. This is also the case for a number of environmental sites such as wastewater samples, where HS⁻ is known to cause corrosion of concrete infrastructure within the sewage industry, whilst the gas H₂S is dangerous at high concentrations and foul smelling at low concentrations.

Methods for the detection of hydrosulfide anions (HS⁻) in water have been limited largely due to the challenges of overcoming the competitive anion hydration in the design of host systems capable of the molecular recognition of anion guest species in aqueous media. Over the years highly charged polycationic receptors, Lewis acidic main group and metal based hosts, together with cyclic peptides and hydrophobic cage-like hosts have been the leading effective strategies for aqueous anion recognition to date.^{1,8,9} Methods for aqueous HS⁻ detection have been largely confined to irreversible chemodosimeter approaches.^{10,11} Only up until recently has a reversible supramolecular host-guest approach to binding HS⁻ been reported in organic solvent media using acyclic bis-urea¹² and tripodal amide receptors,¹³ and only with bambusuril macrocycles in water.^{14,15}

A recent addition to the anion-binding supramolecular toolbox is halogen bonding (XB), a highly directional, attractive interaction between an electron-deficient halogen atom, and a Lewis base.^{16,17} Although the few acyclic and macrocyclic XB receptors reported thus far commonly exhibit superior anion binding affinities, as well as enhanced sensory performance, compared to hydrogen bonding (HB) analogues,¹⁸⁻²¹ their application in aqueous samples such as pure water are notably rare^{20,22}. Thus, there remains a need for efficient anion sensing in aqueous media such as physiological and wastewater samples.

The present invention was devised with the foregoing in mind.

### SUMMARY OF THE INVENTION

According to a first aspect of the present invention there is provided a compound having a structure according to Formula I defined herein.

According to a second aspect of the present invention there is provided a sensor comprising a compound having a structure according to Formula I defined herein.

According to a third aspect of the present invention there is provided a use of a compound according to the first aspect of the present invention or a sensor according to the second aspect of the present invention for detecting the presence or absence of sulphide in a solution.

According to a fourth aspect of the present invention there is provided a method of detecting the presence or absence of sulphide in a solution, as defined herein.

In the first aspect of the invention, it is particularly suitable that Y₁ and Y₂ are not both absent and Y₁ and Y₂ are not both a fluorophore comprising coumarin or BODIPY.

### DETAILED DESCRIPTION OF THE INVENTION

### Definitions

The term “(m-nC)” or “(m-nC) group” used alone or as a prefix, refers to any group having m to n carbon atoms.

The term “alkyl” as used herein refers to straight or branched chain alkyl moieties, typically having 1, 2, 3, 4, 5 or 6 carbon atoms. This term includes reference to groups such as methyl, ethyl, propyl (n-propyl or isopropyl), butyl (n-butyl, sec-butyl or tert-butyl), pentyl, hexyl and the like. In particular, an alkyl may have 1, 2, 3 or 4 carbon atoms.

The term “alkenyl” as used herein refers to straight or branched chain alkenyl moieties, typically having 1, 2, 3, 4, 5 or 6 carbon atoms. The term includes reference to alkenyl moieties containing 1, 2 or 3 carbon-carbon double bonds (C═C). This term includes reference to groups such as ethenyl (vinyl), propenyl (allyl), butenyl, pentenyl and hexenyl, as well as both the cis and trans isomers thereof.

The term “alkynyl” as used herein refers to straight or branched chain alkynyl moieties, typically having 1, 2, 3, 4, 5 or 6 carbon atoms. The term includes reference to alkynyl moieties containing 1, 2 or 3 carbon-carbon triple bonds (C≡C). This term includes reference to groups such as ethynyl, propynyl, butynyl, pentynyl and hexynyl.

The term “alkoxy” as used herein refers to —O-alkyl, wherein alkyl is straight or branched chain and comprises 1, 2, 3, 4, 5 or 6 carbon atoms. In one class of embodiments, alkoxy has 1, 2, 3 or 4 carbon atoms. This term includes reference to groups such as methoxy, ethoxy, propoxy, isopropoxy, butoxy, tert-butoxy, pentoxy, hexoxy and the like.

The term “aryl” or “aromatic” as used herein means an aromatic ring system comprising 6, 7, 8, 9 or 10 ring carbon atoms. Aryl is often phenyl but may be a polycyclic ring system, having two or more rings, at least one of which is aromatic. This term includes reference to groups such as phenyl, naphthyl and the like. A particularly suitable aryl group is phenyl.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom.

The term “halogen” or “halo” as used herein refers to F, Cl, Br or I.

The term “substituted” as used herein in reference to a moiety means that one or more, especially up to 5, more especially 1, 2 or 3, of the hydrogen atoms in said moiety are replaced independently of each other by the corresponding number of the described substituents. The term “optionally substituted” as used herein means substituted or unsubstituted.

It will, of course, be understood that substituents are only at positions where they are chemically possible, the person skilled in the art being able to decide (either experimentally or theoretically) without inappropriate effort whether a particular substitution is possible.

Throughout the entirety of the description and claims of this specification, where subject matter is described herein using the term “comprise” (or “comprises” or “comprising”), the same subject matter instead described using the term “consist of” (or “consists of” or “consisting of”) or “consist essentially of” (or “consists essentially of” or “consisting essentially of”) is also contemplated.

Throughout the description and claims of this specification, the singular encompasses the plural unless the context otherwise requires. In particular, where the indefinite article is used, the specification is to be understood as contemplating plurality as well as singularity, unless the context requires otherwise.

Features described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any of the specific embodiments recited herein. The invention extends to any novel one, or any novel combination, of the features disclosed in this specification (including any accompanying claims, abstract and drawings), or to any novel one, or any novel combination, of the steps of any method or process so disclosed.

### Compounds of the Invention

As described hereinbefore, in accordance with the first aspect of the present invention there is provided a compound having a structure according to Formula I shown below:

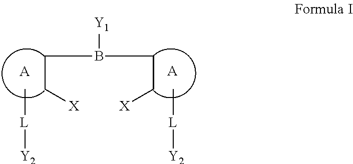

wherein
each X is independently I or Br;
each ring A is independently:
   i) an electron deficient 5- or 6-membered nitrogen-containing heteroaryl that is optionally substituted with one or more groups Rₑ, where each Rₑ is independently selected from (1-6C)alkyl, oxygen and aryl;
   or
   ii) a phenyl that is substituted with one or more groups R_{f}, where each R_{f} is an electron withdrawing group;
each L is independently absent (such that ring A is directly bound to Y₂) or a π-conjugated linker that separates ring A from Y₂ by a distance of 4 atoms or fewer;
Y₁ is absent or is a fluorophore comprising coumarin or BODIPY;
Y₂ is absent or is a fluorophore comprising coumarin or BODIPY;
B is a backbone group of the following structure:

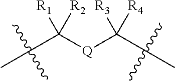

wherein [symbol] denotes the point of attachment to each ring A; and
   i) R₁, R₂, R₃ and R₄ are independently selected from hydrogen, halo, hydroxy, (1-4C)alkyl, (2-4C)alkenyl, (2-4C)alkynyl, (1-4C)haloalkyl and (1-4C)alkoxy; and
   Q is selected from CRₐR_{b}, N⁺RₐR_{b} and NR_{c}, where Rₐ, R_{b} and R_{c} are each independently hydrogen, (1-6C)alkyl or aryl;
   or
   ii) R₁ and R₄ are absent, and R₂ and R₃ are linked to one another, such that when taken in combination with the atoms to which they are attached, they form a 5- or 6-membered heteroaryl or an aryl that is optionally substituted with one or more groups R_{d}, where each R_{d} is independently selected from halo, hydroxy, nitro, cyano, (1-6C)alkyl, (2-6C)alkenyl, (2-6C)alkynyl, (1-6C)haloalkyl, (1-6C)alkoxy, —NR_{d1}R_{d2}, —C(O)—R_{d1}, and —C(O)—OR_{d1}, where R_{d1} and R_{d2} are each independently selected from hydrogen and (1-4C)alkyl; and
   Q is an atom in the 5- or 6-membered heteroaryl ring or the aryl ring;
with the proviso that Y₁ and Y₂ are not both absent; and
wherein the compound of Formula I is substituted with one or more water solubilising groups, Z.

Through rigorous investigations, the inventors have devised new compounds that serve as efficient anion sensors for sensing sulphide anions (e.g. HS⁻) in aqueous solutions such as wastewater and biological samples. The new compounds, which exhibit fluorescence quenching upon binding to sulphide anions, are able to sense sulphide anions in pure water. Importantly, the binding of sulphide anions to the compounds of the invention is completely reversible, thereby representing a step change from traditional irreversible chemodosimeter approaches.

As described above, each X is independently I or Br. In an embodiment, each X is I.

The compounds of the invention are able to bind sulphide anions via the halogen bond donor groups, X. Suitably, the compounds of the invention comprise no more than 2 groups (e.g., I or Br) capable of serving as a halogen bond donor.

Each ring A may be independently an electron deficient 5-membered nitrogen-containing heteroaryl that is optionally substituted with one or more groups Rₑ. The term “electron deficient” will be understood to mean that the electron density per aromatic nucleus of the electron deficient 5- or 6-membered nitrogen-containing heteroaryl is less than the electron density per aromatic nucleus of benzene. Suitably, each ring A is independently an electron deficient 5-membered nitrogen-containing heteroaryl comprising 1, 2 or 3 heteroatoms that is optionally substituted with one or more groups Rₑ. More suitably, each ring A is independently an electron deficient 5-membered nitrogen-containing heteroaryl comprising 1, 2 or 3 nitrogen atoms that is optionally substituted with one or more groups Rₑ. Yet more suitably, each ring A is independently an electron deficient 5-membered nitrogen-containing heteroaryl comprising 2 or 3 nitrogen atoms that is optionally substituted with one or more groups Rₑ. Yet even more suitably, each ring A is independently an electron deficient 5-membered nitrogen-containing heteroaryl comprising 3 nitrogen atoms that is optionally substituted with one or more groups Rₑ. Yet even more suitably, each ring A is independently a triazole or an oxadiazole that is optionally substituted with one or more groups Rₑ. In particularly suitable embodiments, each ring A is independently a triazole that is optionally substituted with one or more groups Rₑ.

It will be understood that each ring A may independently bind to B, X and L (or Y₂ when L is absent) via any atom in the electron deficient 5- or 6-membered nitrogen-containing heteroaryl. For example, it may be that each ring A independently binds to B via a heteroatom (e.g. nitrogen) in the electron deficient 5- or 6-membered nitrogen containing heteroaryl. It may also be that each ring A independently binds to B via a carbon atom in the electron deficient 5- or 6-membered nitrogen containing heteroaryl. It may also be that each ring A independently binds to X via a carbon atom in the electron deficient 5- or 6-membered nitrogen containing heteroaryl. It may also be that each ring A independently binds to L (or Y₂ when L is absent) via a heteroatom (e.g. nitrogen) in the electron deficient 5- or 6-membered nitrogen containing heteroaryl. It may also be that each ring A independently binds to L (or Y₂ when L is absent) via a carbon atom in the electron deficient 5- or 6-membered nitrogen containing heteroaryl.

In certain embodiments, each ring A is independently a triazole that is optionally substituted with one or more groups Rₑ and binds to B, X and L (or Y₂ when L is absent) in the following manner:

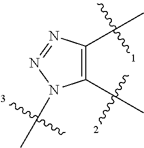 [SMILES: *c1nnn(*)c1*]

wherein
[symbol] ₁ denotes the point of attachment to B;
[symbol] ₂ denotes the point of attachment to X; and
[symbol] ₃ denotes the point of attachment to L (or Y₂ when L is absent).

In certain embodiments, each ring A is independently a triazole that is optionally substituted with one or more groups Rₑ and binds to B, X and L (or Y₂ when L is absent) in the following manner:

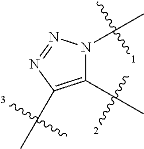 [SMILES: *c1nnn(*)c1*]

wherein
[symbol] ₁ denotes the point of attachment to B;
[symbol] ₂ denotes the point of attachment to X; and
[symbol] ₃ denotes the point of attachment to L (or Y₂ when L is absent).

In certain embodiments, each ring A is independently an oxadiazole that is optionally substituted with one or more groups Rₑ and binds to B, X and L (or Y₂ when L is absent) in the following manner:

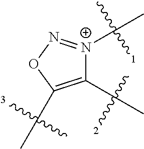 [SMILES: *c1on[n+](*)c1*]

wherein
[symbol] ₁ denotes the point of attachment to B;
[symbol] ₂ denotes the point of attachment to X; and
[symbol] ₃ denotes the point of attachment to L (or Y₂ when L is absent).

Each Rₑ may be independently selected from (1-4C)alkyl, oxygen and aryl. Suitably, each Rₑ is independently selected from (1-3C)alkyl, oxygen and 6-membered aryl. More suitably, each Rₑ is independently selected from methyl, oxygen and phenyl.

In certain embodiments, each ring A is independently a triazolium and binds to B, X and L (or Y₂ when L is absent) in the following manner:

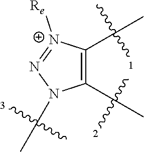

wherein
[symbol] ₁ denotes the point of attachment to B;
[symbol] ₂ denotes the point of attachment to X;
[symbol] ₃ denotes the point of attachment to L (or Y₂ when L is absent); and
Rₑ is defined anywhere herein.

In certain embodiments, each ring A is independently a triazolium and binds to B, X and L (or Y₂ when L is absent) in the following manner:

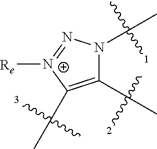

wherein
[symbol] ₁ denotes the point of attachment to B;
[symbol] ₂ denotes the point of attachment to X;
[symbol] ₃ denotes the point of attachment to L (or Y₂ when L is absent); and
Rₑ is defined anywhere herein.

In certain embodiments, each ring A is independently a triazole N-oxide and binds to B, X and L (or Y₂ when L is absent) in the following manner:

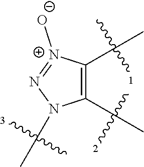 [SMILES: *c1c(*)[n+]([O-])nn1*]

wherein
[symbol] ₁ denotes the point of attachment to B;
[symbol] ₂ denotes the point of attachment to X; and
[symbol] ₃ denotes the point of attachment to L (or Y₂ when L is absent).

In certain embodiments, each ring A is independently a triazole N-oxide and binds to B, X and L (or Y₂ when L is absent) in the following manner:

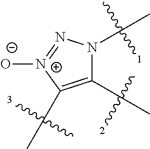 [SMILES: *c1c(*)[n+]([O-])nn1*]

wherein
[symbol] ₁ denotes the point of attachment to B;
[symbol] ₂ denotes the point of attachment to X; and
[symbol] ₃ denotes the point of attachment to L (or Y₂ when L is absent).

In certain embodiments, each ring A may be independently unsubstituted.

It will be understood that in certain embodiments wherein each ring A is independently an electron deficient 5- or 6-membered nitrogen-containing heteroaryl that is substituted with one or more groups Rₑ, substitution may only take place where chemically possible. For example, it may be that each ring A is independently an electron deficient 5- or 6-membered nitrogen-containing heteroaryl that is substituted with one group Rₑ on a heteroatom (e.g. nitrogen), thereby forming a positively charged triazolium analogue. Similarly, it may be that each ring A is independently an electron deficient 5- or 6-membered nitrogen-containing heteroaryl that is substituted with one group Rₑ on a heteroatom (e.g. nitrogen), thereby forming a neutral triazole N-oxide analogue (i.e. positively charged nitrogen atom and negatively charged oxygen). Suitably, each ring A is independently an electron deficient 5- or 6-membered nitrogen-containing heteroaryl (i.e. not substituted by one or more groups Rₑ).

As described above, each ring A may be independently a phenyl that is substituted with one or more groups R_{f}, where each R_{f} is an electron withdrawing group. The term “electron withdrawing group” when used in the context of each ring A will be understood to mean a group which pulls electron density away from the phenyl ring (i.e. removing electron density from the T-system). Such groups will be familiar to one of ordinary skill in the art. Each ring A may be independently a phenyl that is substituted with 1, 2 or 3 groups R_{f}. Suitably, each ring A is independently a phenyl that is substituted with one or two groups R_{f}. More suitably, each ring A is independently a phenyl that is substituted with one group R_{f}.

Most suitably, both rings A are identical.

Each R_{f} may be independently selected from fluoro, chloro, nitro, cyano, (1-3C)haloalkyl, —C(O)—R_{f1} and —C(O)O—R_{f1}, where R_{f1} is independently selected from hydrogen and (1-2C)alkyl. Suitably, each R_{f} is independently selected from fluoro, nitro, cyano, —C(O)—R_{f1} and —C(O)O—R_{f1}, where R_{f1} is independently selected from hydrogen and (1-2C)alkyl (e.g. methyl). More suitably, each R_{f} is independently selected from fluoro, chloro, nitro and cyano.

Each L may be independently absent (such that ring A is directly bound to Y₂) or a x-conjugated linker that separates ring A from Y₂ by a distance of 2-4 atoms (e.g. an ethenylene linker or a phenylene linker). Suitably, each L is independently absent or a π-conjugated linker that separates ring A from Y₂ by a distance of 4 atoms or less, wherein the π-conjugated linker comprises fewer than 20 atoms in total. More suitably, each L is independently absent or a T-conjugated linker that separates ring A from Y₂ by a distance of 4 atoms or less, wherein the T-conjugated linker comprises fewer than 10 atoms in total. Yet more suitably, each L is absent (such that ring A is directly bound to Y₂).

Most suitably, both groups L are identical.

Y₁ is absent or is a fluorophore comprising coumarin or BODIPY, and Y₂ is absent or is a fluorophore comprising coumarin or BODIPY, with the proviso that Y₁ and Y₂ are not both absent. Coumarin and BODIPY, including substituted analogues thereof, are known to exhibit fluorescence.

Y₁ may be absent or may be a fluorophore comprising coumarin or BODIPY. Suitably, Y₁ is absent.

Y₂ may be absent or may be a fluorophore comprising coumarin or BODIPY. Suitably, Y₂ is a fluorophore comprising coumarin or BODIPY.

As described above, Y₁ and Y₂ are not both absent. It is particularly suitable that Y₁ and Y₂ are not both absent and Y₁ and Y₂ are not both a fluorophore comprising coumarin or BODIPY. Even more suitably, Y₁ is absent and Y₂ is a fluorophore comprising coumarin or BODIPY.

Most suitably, both groups Y₂ are identical.

The structures of coumarin and BODIPY are as follows:

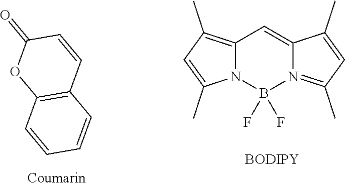

Thus, when present, Y₁ and Y₂ may be coumarin or BODIPY as depicted above.

In certain particularly suitable embodiments, Y₁ is absent and Y₂ is a fluorophore selected from:

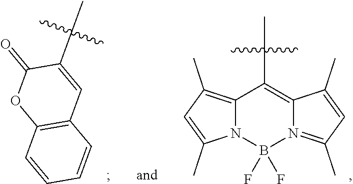

wherein [symbol] denotes the point of attachment to L (or ring A when L is absent).

As described above, B is a backbone group of the following structure:

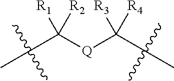

wherein [symbol] denotes the point of attachment to each ring A.

R₁, R₂, R₃ and R₄ may be independently selected from hydrogen, chloro, fluoro, hydroxy, (1-3C)alkyl, (2-3C)alkenyl, (2-3C)alkynyl, (1-3C)haloalkyl and (1-3C)alkoxy. Suitably, R₁, R₂, R₃ and R₄ are independently selected from hydrogen, (1-3C)alkyl, (1-3C)haloalkyl and (1-3C)alkoxy. More suitably, R₁, R₂, R₃ and R₄ are independently selected from hydrogen and (1-3C)alkyl. Even more suitably, R₁, R₂, R₃ and R₄ are independently selected from hydrogen.

In certain embodiments wherein R₁, R₂, R₃ and R₄ are independently selected from hydrogen, halo, hydroxy, (1-4C)alkyl, (2-4C)alkenyl, (2-4C)alkynyl, (1-4C)haloalkyl and (1-4C)alkoxy, Q is selected from CRₐR_{b}, N⁺RₐR_{b} and NR_{c}, where Rₐ, R_{b} and R_{c} are each independently hydrogen, (1-3C)alkyl or phenyl. Suitably, Q is selected from CRₐR_{b}, N⁺RₐR_{b} and NR_{c}, where Rₐ, R_{b} and R_{c} are each independently hydrogen, (1-3C)alkyl or phenyl. More suitably, Q is selected from N⁺RₐR_{b} and NR_{c}, where Rₐ, R_{b} and R_{c} are each independently hydrogen or methyl. Even more suitably, Q is selected from N⁺RₐR_{b} and NR_{c}, where Rₐ, R_{b} and R_{c} are each independently hydrogen. Yet even more suitably, Q is NR_{c}, where R_{c} is hydrogen.

In certain embodiments, B is a backbone group having a structure selected from the following:

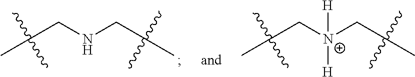

wherein [symbol] denotes the point of attachment to each ring A.

Alternatively, R₁ and R₄ may be absent, and R₂ and R₃ may be linked to one another, such that when taken in combination with the atoms to which they are attached, they form a 5- or 6-membered heteroaryl or a phenyl that is optionally substituted with one or more groups R_{d}. In such instances, Q is a ring atom within the 5- or 6-membered heteroaryl or phenyl. Suitably, R₁ and R₄ are absent, and R₂ and R₃ are linked to one another, such that when taken in combination with the atoms to which they are attached, they form a 6-membered nitrogen-containing heteroaryl or a phenyl that is optionally substituted with one or more groups R_{d}. More suitably, R₁ and R₄ are absent, and R₂ and R₃ are linked to one another, such that when taken in combination with the atoms to which they are attached, they form a 6-membered heteroaryl containing 1 or 2 nitrogen atoms or a phenyl that is optionally substituted with one or more groups R_{d}. Even more suitably, R₁ and R₄ are absent, and R₂ and R₃ are linked to one another, such that when taken in combination with the atoms to which they are attached, they form a pyrimidine, a pyridine or a phenyl that is optionally substituted with one or more groups R_{d}.

Each Rₐ may be independently selected from halo, hydroxy, nitro, cyano, (1-3C)alkyl, (2-3C)alkenyl, (2-3C)alkynyl, (1-3C)haloalkyl, (1-3C)alkoxy, —NR_{d1}R_{d2}, —C(O)—R_{d1}, and —C(O)—OR_{d1}, where R_{d1} and R_{d2} are each independently selected from hydrogen and (1-4C)alkyl. Suitably. each R_{d} is independently selected from nitro, (1-3C)alkyl, —C(O)—R_{d1}, and —C(O)—OR_{d1}, where R_{d1} is selected from hydrogen and (1-2C)alkyl. More suitably, each R_{d} is independently selected from nitro, (1-2C)alkyl, and —C(O)—R_{d1}, where R_{d1} is hydrogen. Even more suitably, each R_{d} is independently selected from (1-2C)alkyl.

In certain embodiments wherein R₁ and R₄ are absent, and R₂ and R₃ are linked to one another, such that when taken in combination with the atoms to which they are attached, they form a 5- or 6-membered heteroaryl or an aryl that is optionally substituted with one or more groups R_{d}, Q is an atom in the 5- or 6-membered heteroaryl ring or the aryl ring selected from a carbon atom and a nitrogen atom.

In certain embodiments, R₁ and R₄ are absent, and R₂ and R₃ are linked to one another, such that when taken in combination with the atoms to which they are attached, they form a backbone group B having one of the following structures:

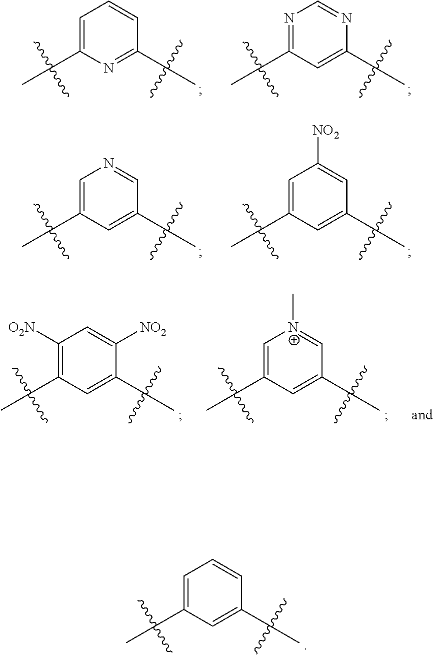

wherein [symbol] denotes the point of attachment to each ring A.

In particularly suitable embodiments, R₁ and R₄ are absent, and R₂ and R₃ are linked to one another, such that when taken in combination with the atoms to which they are attached, they form a backbone group B having one of the following structures:

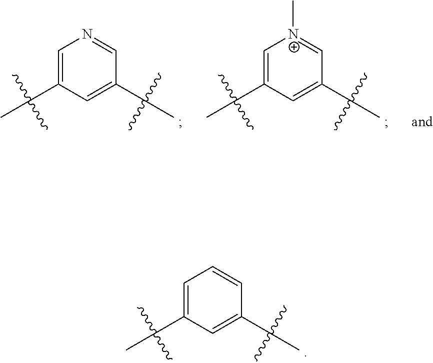

wherein [symbol] denotes the point of attachment to each ring A.

It will be understood that the compound of Formula I is substituted with one or more water solubilising groups, Z, in any position on said compound which is chemically possible. For example, the one or more water solubilising groups, Z, may be bound to Y₁ or Y₂, when present. Alternatively (or additionally), the one or more water solubilising groups, Z, may be bound to B (e.g. when Y₁ is absent). Alternatively (or additionally), the one or more water solubilising groups, Z, may be bound to each ring A (e.g. when Y₂ is absent).

In certain embodiments, the compound of Formula I is substituted with one water solubilising group, Z, being bound to the backbone B. In such embodiments, Y₂ is suitably absent and Y₁ is suitably a fluorophore comprising BODIPY.

In certain particularly suitable embodiments, the compound of Formula I is substituted with two water solubilising groups, Z, each being bound to separate Y₁ groups (i.e. one Z per Y₁). In such embodiments, Y₂ is suitably absent and Y₁ is suitably a fluorophore comprising coumarin.

The one or more water solubilising groups, Z, may comprise i) one or more poly(2-4C)alkylene glycol groups, each comprising 2-10 repeating units, or ii) one or more carboxylate or carboxylate salt groups. Suitably, the one or more water solubilising groups, Z, may comprise i) one or more poly(2-3C)alkylene glycol groups, each comprising 2-8 repeating units, or ii) two or more carboxylate or carboxylate salt groups. More suitably, the one or more water solubilising groups, Z, comprises i) two or more polyethylene glycol groups, each comprising 3-6 repeating units, or ii) three or more carboxylate or carboxylate salt groups. In particular embodiments, the one or more water solubilising groups, Z, is independently selected from one of the following structures:

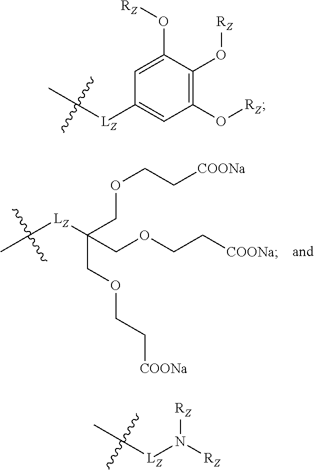

wherein [symbol] denotes the point of attachment of Z within the compound of Formula I (e.g. the point of attachment to Y₂ or B);
L_{Z} is absent (i.e. bond) or is a linking group containing 15 atoms or fewer in total; and
R_{z} is a polyethylene glycol group, each comprising 2-10 (or 2-8 or 3-6) repeating ethylene glycol units.

Suitably, L_{Z} is absent or is a linking group-(L_{Z1})-(L_{Z2})-(L_{Z3})—, where L_{Z1} and L_{Z3} are independently absent or a (1-4C)alkylene or (2-4C)alkenylene group, and L_{Z2} is absent or is a group —O—, —NH—, —OC(O)—, —C(O)O—, —C(O)—, —NHC(O)— or —C(O) NH—.

Most suitably, all groups Z are identical.

Particularly suitable combinations of groups Y₁, Y₂, A, B, X, L and Z (and any sub-definitions thereof) are outlined in the following embodiments.

In certain particular embodiments:
X is I;
L is absent; and
each ring A is independently a triazole or an oxadiazole that is optionally substituted with one or more groups Rₑ, where each Rₑ is independently selected from methyl, oxygen and phenyl;

In certain particular embodiments:
L is absent;
each ring A is independently a triazole or an oxadiazole that is optionally substituted with one or more groups Rₑ, where each Rₑ is independently selected from methyl, oxygen and phenyl; and
each Z, comprises i) one or more poly(2-3C)alkylene glycol groups, each comprising 2-8 repeating units, or ii) two or more carboxylate or carboxylate salt groups.

In certain particular embodiments:
X is I;
L is absent;
each ring A is independently (i) a triazole or an oxadiazole that is optionally substituted with one or more groups Rₑ, where each Rₑ is independently selected from methyl, oxygen and phenyl, or (ii) a phenyl that is substituted with one or more groups R_{f}; and
Y₁ is absent and Y₂ is a fluorophore selected from:

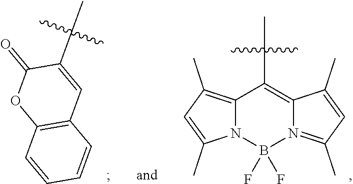

wherein [symbol] denotes the point of attachment to ring A.

In certain particular embodiments:
X is I;
L is absent;
each ring A is independently a triazole or an oxadiazole that is optionally substituted with one or more groups Rₑ, where each Rₑ is independently selected from methyl, oxygen and phenyl;
each Z is independently selected from one of the following structures:

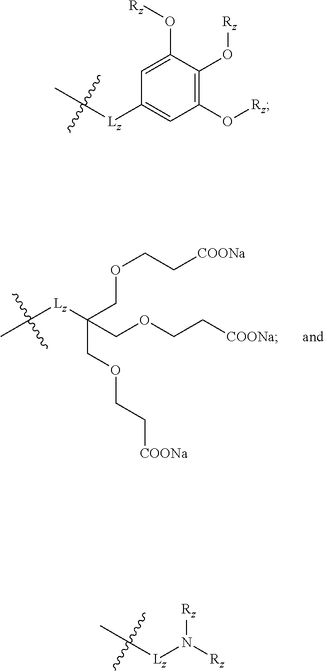

wherein [symbol] denotes the point of attachment of Z within the compound of Formula I (e.g. the point of attachment to Y² or B);
L_{Z} is absent or is a linking group containing 15 atoms or fewer;
R_{z} is a polyethylene glycol group, each comprising 2-10 (or 2-8 or 3-6) repeating units; and
Y₁ is absent and Y₂ is a fluorophore selected from:

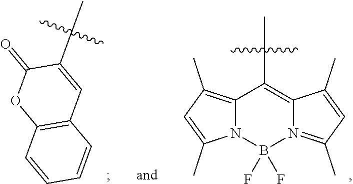

wherein [symbol] denotes the point of attachment to ring A.

In certain particular embodiments:
X is I;
L is absent;
each ring A is independently a triazole or an oxadiazole that is optionally substituted with one or more groups Rₑ, where each Rₑ is independently selected from methyl, oxygen and phenyl; and
B is a backbone group having a structure selected from the following:

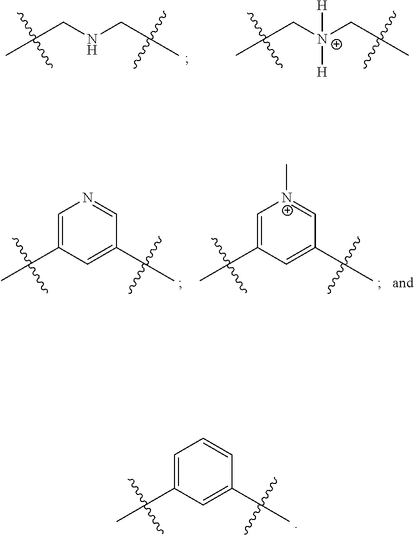

wherein [symbol] denotes the point of attachment to each ring A.

In certain particular embodiments:
X is I;
L is absent;
each ring A is independently (i) a triazole or an oxadiazole that is optionally substituted with one or more groups Rₑ, where each Rₑ is independently selected from methyl, oxygen and phenyl, or (ii) a phenyl that is substituted with one or more groups R_{f};
Y₁ is absent and Y₂ is a fluorophore selected from:

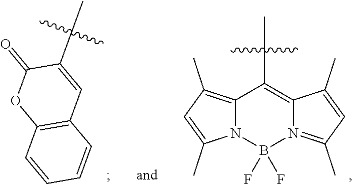

wherein [symbol] denotes the point of attachment to ring A;
B is a backbone group having a structure selected from the following:

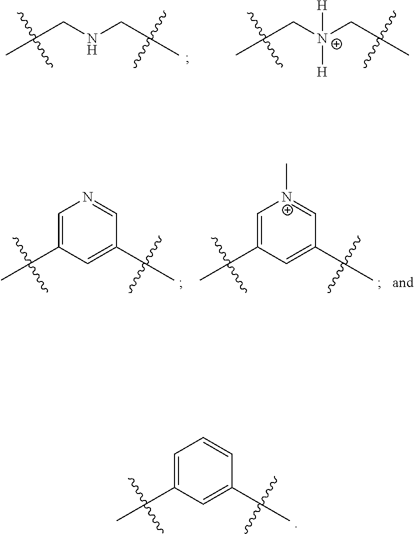

wherein [symbol] denotes the point of attachment to each ring A; and
each Z is independently selected from one of the following structures:

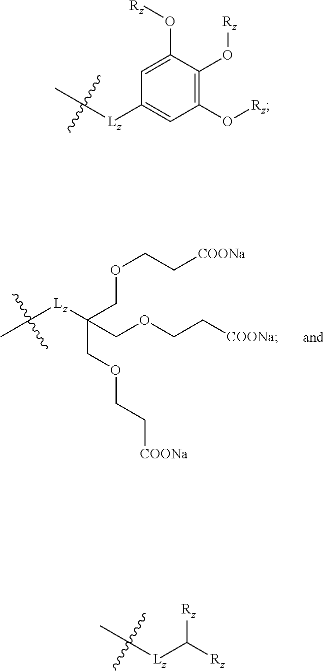

wherein [symbol] denotes the point of attachment of Z within the compound of Formula I (e.g. the point of attachment to Y² or B);
L_{Z} is absent or is a linking group containing 15 atoms or fewer; and
R_{z} is a polyethylene glycol group, each comprising 2-10 (or 2-8 or 3-6) repeating units.

In certain particular embodiments:
X is I;
L is absent;
each ring A is independently a triazole or an oxadiazole that is optionally substituted with one or more groups Rₑ, where each Rₑ is independently selected from methyl, oxygen and phenyl;
Y₁ is absent and Y₂ is a fluorophore:

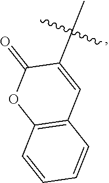

wherein [symbol] denotes the point of attachment to ring A; and
the compound of Formula I is substituted with two water solubilising groups, Z, each being bound to separate Y¹ groups, where Z comprises i) one or more poly(2-3C)alkylene glycol groups, each comprising 2-8 repeating units, or ii) two or more carboxylate or carboxylate salt groups.

In certain particular embodiments:
X is I;
L is absent;
each ring A is independently a triazole or an oxadiazole that is optionally substituted with one or more groups Rₑ, where each Rₑ is independently selected from methyl, oxygen and phenyl;
Y₁ is absent and Y₂ is a fluorophore:

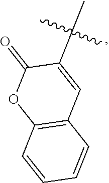

wherein [symbol] denotes the point of attachment to ring A;
B is a backbone group having a structure selected from the following:

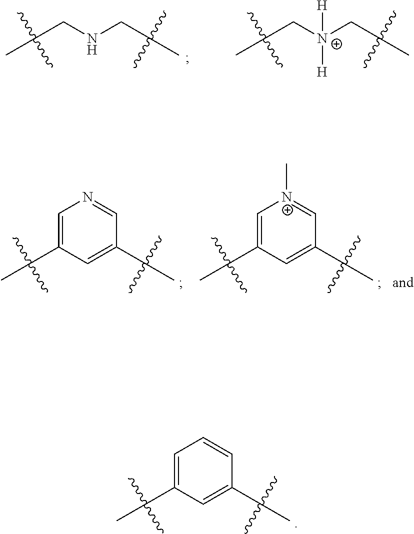

wherein [symbol] denotes the point of attachment to each ring A; and
the compound of Formula I is substituted with two water solubilising groups, Z, each being bound to separate Y¹ groups, where Z comprises i) one or more poly(2-3C)alkylene glycol groups, each comprising 2-8 repeating units, or ii) two or more carboxylate or carboxylate salt groups.

In certain particular embodiments:
X is I;
L is absent;
each ring A is independently a triazole or an oxadiazole that is optionally substituted with one or more groups Rₑ, where each Rₑ is independently selected from methyl, oxygen and phenyl;
Y₁ is absent and Y₂ is a fluorophore:

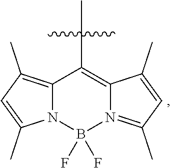

wherein [symbol] denotes the point of attachment to ring A; and
the compound of Formula I is substituted with one water solubilising groups, Z, bound to the backbone B, where Z comprises i) one or more poly(2-3C)alkylene glycol groups, each comprising 2-8 repeating units, or ii) two or more carboxylate or carboxylate salt groups.

In certain particular embodiments:
X is I;
L is absent;
each ring A is independently a triazole or an oxadiazole that is optionally substituted with one or more groups Rₑ, where each Rₑ is independently selected from methyl, oxygen and phenyl;
Y₁ is absent and Y₂ is a fluorophore:

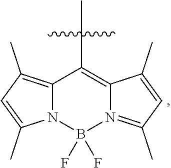

wherein [symbol] denotes the point of attachment to ring A;
B is a backbone group having a structure selected from the following:

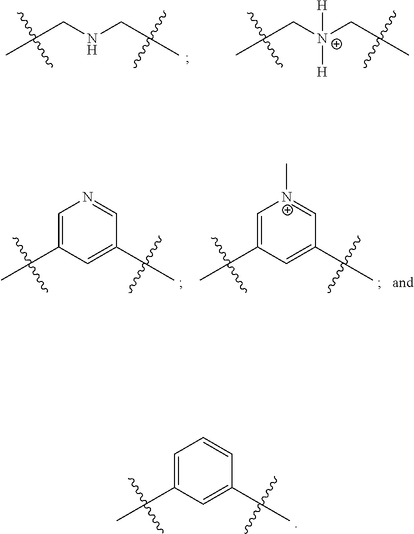

wherein [symbol] denotes the point of attachment to each ring A; and
the compound of Formula I is substituted with one water solubilising group, Z, bound to the backbone B, where Z comprises i) one or more poly(2-3C)alkylene glycol groups, each comprising 2-8 repeating units, or ii) two or more carboxylate or carboxylate salt groups.

In any of the above-outlined particular embodiments, A is most suitably a triazole.

In any of the above-outlined particular embodiments, L is most suitably absent.

In any of the above-outlined particular embodiments, each Z is most suitably selected from one of the following structures:

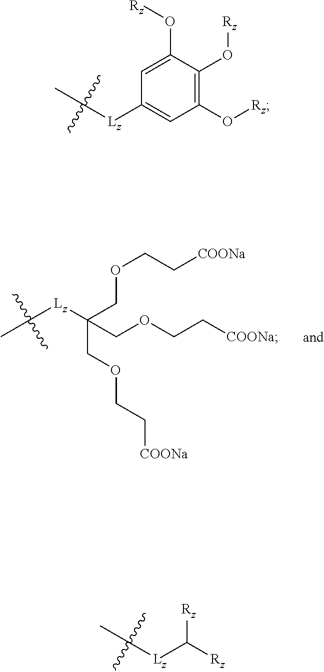

wherein [symbol] denotes the point of attachment of Z within the compound of Formula I (e.g. the point of attachment to Y² or B);
L_{Z} is absent or is a linking group-(L_{Z1})-(L_{Z2})-(L_{Z3})—, where L_{Z1} and L_{Z3} are independently absent or a (1-4C)alkylene or (2-4C)alkenylene group, and L_{Z2} is absent or is a group —O—, —NH—, —OC(O)—, —C(O)O—, —C(O)—, —NHC(O)— or —C(O)NH—, wherein L_{Z}, when present, contains 15 atoms or fewer in total; and
R_{z} is a polyethylene glycol group, each comprising 3-6 repeating units.

In any of the above-outlined particular embodiments, both A groups most suitably have identical definitions, the same being true of both X groups, both L groups and both Y₂ groups.

In a particular class of embodiments, the compound of Formula I has a structure according to Formula Ia (which is a sub-definition of Formula I), shown below:

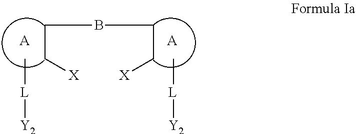

wherein
the compound of Formula Ia is substituted with one or more water solubilising groups, Z; and
Y₂, B, Z, A, L and X, and any associated sub-groups, have any of the definitions outlined hereinbefore in relation to Formula I.

Formula Ia is a sub-set of Formula I. It will therefore be understood that the particular combinations of groups B, A, Z, L, X and Y₂ (and any sub-definitions thereof) outlined hereinbefore in embodiments of compounds of Formula I may also be applicable to the compounds of Formula Ia.

In a particular class of embodiments, the compound of Formula I has a structure according to Formula Ib (which is a sub-definition of Formula I), shown below:

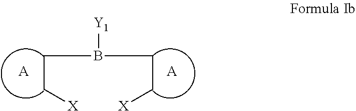

wherein
the compound of Formula Ia is substituted with one or more water solubilising groups, Z; and
Y₁, B, Z, A and X, and any associated sub-groups, have any of the definitions outlined hereinbefore in relation to Formula I.

Formula Ib is a sub-set of Formula I. It will therefore be understood that the particular combinations of groups B, A, Z, X and Y₂ (and any sub-definitions thereof) outlined hereinbefore in embodiments of compounds of Formula I may also be applicable to the compounds of Formula Ib.

In a particular class of embodiments, the compound of Formula I has a structure according to Formula II (which is a sub-definition of Formula I), shown below:

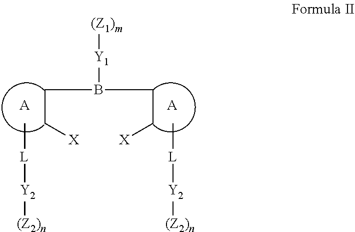

wherein B, A, L, X, Y₁ and Y₂, and any associated sub-groups, have any of the definitions outlined hereinbefore;
Z₁ and Z₂ are each independently a water solubilising group having any of the definitions outlined hereinbefore in relation to Z;
m is 0, 1 or 2; and
n is 0, 1 or 2;
with the proviso that m and n are not both 0.

m may be 0 or 1. Suitably m is 0.

n may be 0 or 1. Suitably n is 1.

Most suitably, both n groups are identical.

Formula II is a sub-set of Formula I. It will therefore be understood that the particular combinations of groups B, A, L, X, Y₁ and Y₂ (and any sub-definitions thereof) outlined hereinbefore in embodiments of compounds of Formula I may also be applicable to the compounds of Formula II. The following paragraphs outline further embodiments of compounds of Formula II.

In certain embodiments of Formula II:
m is 0 and n is 1; and
Y¹ is absent and Y₂ is a fluorophore:

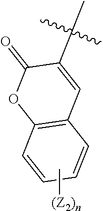

wherein [symbol] denotes the point of attachment to L (or ring A when L is absent).

In certain embodiments of Formula II:
m is 0 and n is 1; and
Y₁ is absent and Y₂ is a fluorophore:

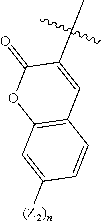

wherein [symbol] denotes the point of attachment to L (or ring A when L is absent).

In certain embodiments of Formula II:
m is 1 and n is 0; and
Y₁ is absent and Y₂ is a fluorophore:

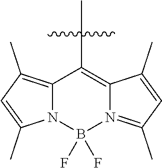

wherein [symbol] denotes the point of attachment to L (or ring A when L is absent).

In certain particular embodiments of Formula II:
(i) m is 0, n is 1, Y₁ is absent and Y₂ is a fluorophore:

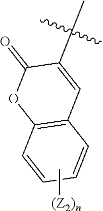

wherein [symbol] denotes the point of attachment to ring A; or
(ii) m is 1, n is 0, Y₁ is absent and Y₂ is a fluorophore:

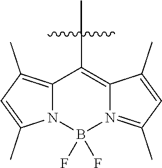

wherein [symbol] denotes the point of attachment to ring A
L is absent;
each ring A is independently (i) a triazole or an oxadiazole that is optionally substituted with one or more groups Rₑ, where each Rₑ is independently selected from methyl, oxygen and phenyl, or (ii) a phenyl that is substituted with one or more groups R_{f}, and
each Z₁ and Z₂, when present, is a water-solubilising group comprising i) one or more poly(2-3C)alkylene glycol groups, each comprising 2-8 repeating units, or ii) two or more carboxylate or carboxylate salt groups.

In certain particular embodiments of Formula II:
(i) m is 0, n is 1, Y¹ is absent and Y₂ is a fluorophore:

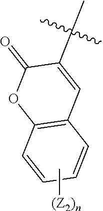

wherein [symbol] denotes the point of attachment to ring A; or
(ii) m is 1, n is 0, Y¹ is absent and Y₂ is a fluorophore:

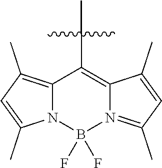

wherein [symbol] denotes the point of attachment to ring A;
L is absent;
each ring A is independently (i) a triazole or an oxadiazole that is optionally substituted with one or more groups Rₑ, where each Rₑ is independently selected from methyl, oxygen and phenyl, or (ii) a phenyl that is substituted with one or more groups R_{f};
B is a backbone group having a structure selected from the following:

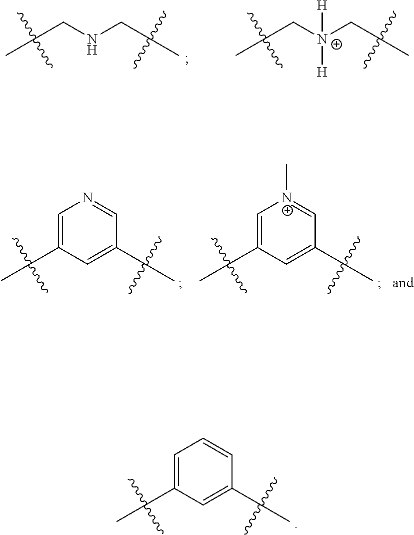

wherein [symbol] denotes the point of attachment to each ring A; and
each Z₁ and Z₂, when present, is a water-solubilising group comprising i) one or more poly(2-3C)alkylene glycol groups, each comprising 2-8 repeating units, or ii) two or more carboxylate or carboxylate salt groups.

In certain particular embodiments of Formula II:
(i) m is 0, n is 1, Y¹ is absent and Y₂ is a fluorophore:

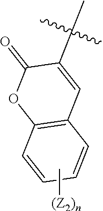

wherein [symbol] denotes the point of attachment to ring A; or
(ii) m is 1, n is 0, Y¹ is absent and Y₂ is a fluorophore:

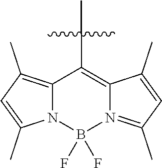

wherein [symbol] denotes the point of attachment to ring A;
L is absent;
each ring A is independently a triazole or an oxadiazole that is optionally substituted with one or more groups Rₑ, where each Rₑ is independently selected from methyl, oxygen and phenyl;
B is a backbone group having a structure selected from the following:

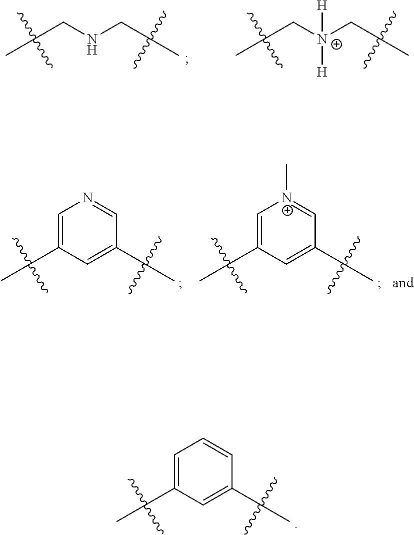

wherein [symbol] denotes the point of attachment to each ring A; and
Z₁ and Z₂, when present, are selected from one of the following structures:

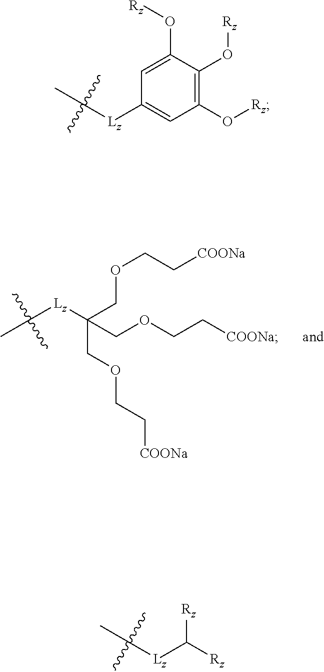

L_{Z} is absent or is a linking group containing 15 atoms or fewer; and
R_{z} is a polyethylene glycol group, each comprising 2-10 (or 2-8 or 3-6) repeating units.

In any of the above-outlined further embodiments of Formula II, A is most suitably a triazole.

In any of the above-outlined further embodiments of Formula II, L is most suitably absent.

In any of the above-outlined further embodiments of Formula II, Z₁ and Z₂, when present, are most suitably selected from one of the following structures:

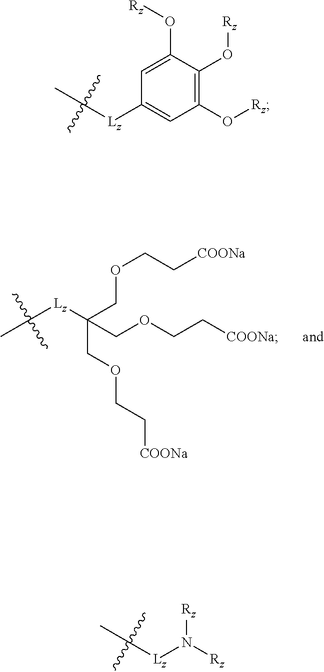

L_{Z} is absent or is a linking group-(L_{Z1})-(L_{Z2})-(L_{Z3})—, where L_{Z1} and L_{Z2} are independently absent or a (1-4C)alkylene or (2-4C)alkenylene group, and L_{Z2} is absent or is a group —O—, —NH—, —OC(O)—, —C(O)O—, —C(O)—, —NHC(O)— or —C(O)NH—, wherein L_{Z}, when present, contains 15 atoms or fewer in total; and
R_{z} is a polyethylene glycol group, each comprising 3-6 repeating units.

In any of the above-outlined further embodiments of Formula II, both A groups most suitably have identical definitions, the same being true of both X groups, both L groups, both Z₂ groups, both n groups and both Y₂ groups.

In a particular class of embodiments, the compound of Formula I has a structure according to Formula IIa (which is a sub-definition of Formula II), shown below:

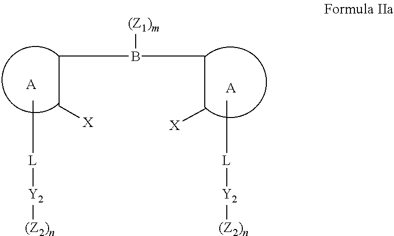

wherein Y₂, m, n, Z₁, Z₂, B, A, L and X, and any associated sub-groups, have any of the definitions outlined hereinbefore in relation to Formula I and any sub-formula thereof.

Formula IIa is a sub-set of Formula I and II. It will therefore be understood that the particular combinations of groups B, A, L, X, m, n, Z₁, Z₂ and Y₂ (and any sub-definitions thereof) outlined hereinbefore in embodiments of compounds of Formula I and II may also be applicable to the compounds of Formula IIa.

In a particular class of embodiments, the compound of Formula I has a structure according to Formula IIa-i, IIa-ii, IIa-iii or IIa-iv (which are sub-definitions of Formula IIa), shown below:

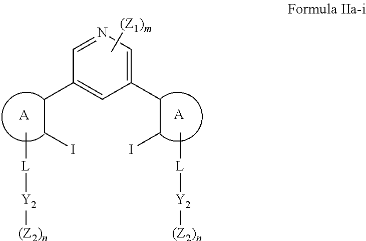

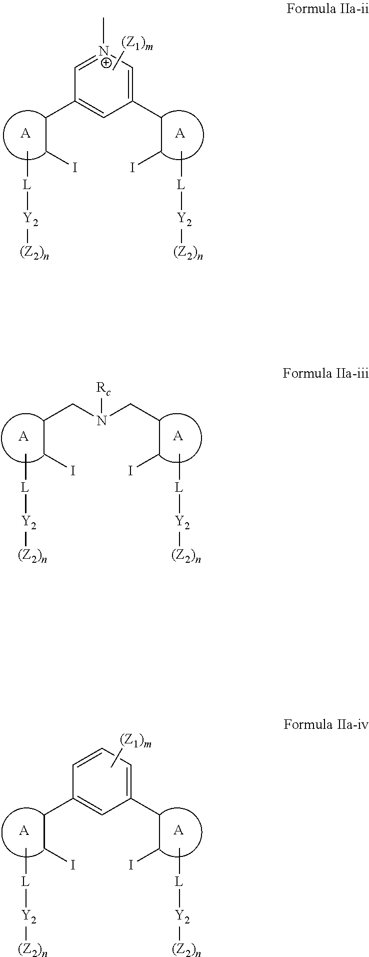

wherein Y₂, m, n, R_{c}, Z₁, Z₂, A and L, and any associated sub-groups, have any of the definitions outlined hereinbefore in relation to Formula I and any sub-formula thereof.

Within Formulae IIa-i to IIa-iv, R_{c} is suitably hydrogen or methyl.

Formulae IIa-i to IIa-iv are sub-sets of Formula I and II. It will therefore be understood that the particular combinations of groups A, L, m, n, Z₁, Z₂ and Y₂ (and any sub-definitions thereof) outlined hereinbefore in embodiments of compounds of Formula I and II may also be applicable to the compounds of Formula IIa-i to IIa-iv.

In a particular class of embodiments, the compound of Formula I has a structure according to Formula IIa-v (which is a sub-definition of Formula IIa), shown below:

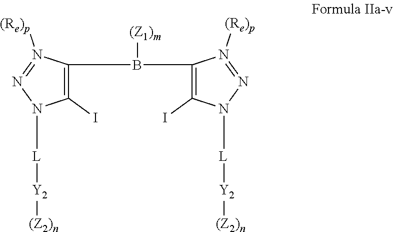

wherein
p is 0 or 1 (it being understood that when p is 1, the N atom to which Rₑ is attached carries a positive charge); and
Y₂, m, n, Rₑ, Z₁, Z₂, B and L, and any associated sub-groups, have any of the definitions outlined hereinbefore in in relation to Formula I and any sub-formula thereof.

Within Formula IIa-v, Y₂ is most suitably a fluorophore comprising coumarin.

Within Formula IIa-v, p may be 1. Suitably, p is 0. Both p groups are suitably identical.

Within Formula IIa-v, each Rₑ may be independently selected from (1-6C)alkyl, oxygen and aryl. Suitably, each Rₑ is independently selected from (1-3C)alkyl, oxygen and phenyl. More suitably, each Rₑ is oxygen. When present, both Rₑ are suitably identical.

Formula IIa-v is a sub-set of Formula I and II. It will therefore be understood that the particular combinations of groups B, L, m, n, Z₁, Z₂ and Y₂ (and any sub-definitions thereof) outlined hereinbefore in embodiments of compounds of Formula I and II may also be applicable to the compounds of Formula IIa-v.

In a particular class of embodiments, the compound of Formula I has a structure according to Formula IIa-vi (which is a sub-definition of Formula IIa), shown below:

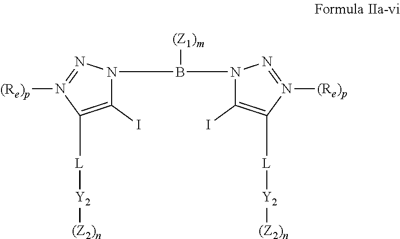

wherein
p is 0 or 1 (it being understood that when p is 1, the N atom to which Rₑ is attached carries a positive charge); and
Y₂, m, n, Rₑ, Z₁, Z₂, B and each L, and any associated sub-groups, have any of the definitions outlined hereinbefore in in relation to Formula I and any sub-formula thereof.

Within Formula IIa-vi, Y₂ is most suitably a fluorophore comprising BODIPY.

Within Formula IIa-vi, p may be 1. Suitably, p is 0. Both p groups are suitably identical.

Within Formula IIa-vi, each Rₑ may be independently selected from (1-6C)alkyl, oxygen and aryl. Suitably, each Rₑ is independently selected from (1-3C)alkyl, oxygen and phenyl. More suitably, each Rₑ is oxygen. When present, both Rₑ are suitably identical.

Formula IIa-vi is a sub-set of Formula I and II. It will therefore be understood that the particular combinations of groups B, L, m, n, Z₁, Z₂ and Y₂ (and any sub-definitions thereof) outlined hereinbefore in embodiments of compounds of Formula I and II may also be applicable to the compounds of Formula IIa-v.

In a particular class of embodiments, the compound of Formula I has a structure according to Formula IIb (which is a sub-definition of Formula II), shown below:

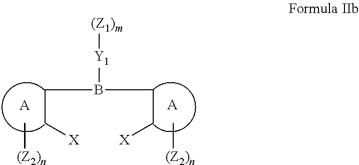

wherein
Y₁, m, n, Z₁, Z₂, B, A and X, and any associated sub-groups, have any of the definitions outlined hereinbefore in relation to Formula I or any sub-formula thereof.

Formula IIb is a sub-set of Formula I and II. It will therefore be understood that the particular combinations of groups B, L, A, X, m, n, Z₁, Z₂ and Y₁ (and any sub-definitions thereof) outlined hereinbefore in embodiments of compounds of Formula I and II may also be applicable to the compounds of Formula IIb.

In a particular class of embodiments, the compound of Formula I has a structure according to Formula IIb-i or IIb-ii (which are sub-definitions of Formula IIb), shown below:

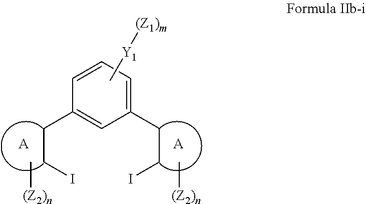

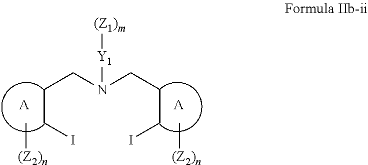

wherein
Y₁, m, n, Z₁, Z₂, B, A and X, and any associated sub-groups, have any of the definitions outlined hereinbefore in relation to Formula I or any sub-formula thereof.

Formula IIb-i and IIb-ii are sub-sets of Formula I and II. It will therefore be understood that the particular combinations of groups L, A, X, m, n, Z₁, Z₂ and Y₁ (and any sub-definitions thereof) outlined hereinbefore in embodiments of compounds of Formula I and II may also be applicable to the compounds of Formula IIb-i and IIb-ii.

In certain embodiments, the compound of Formula I is selected from any one of the following:

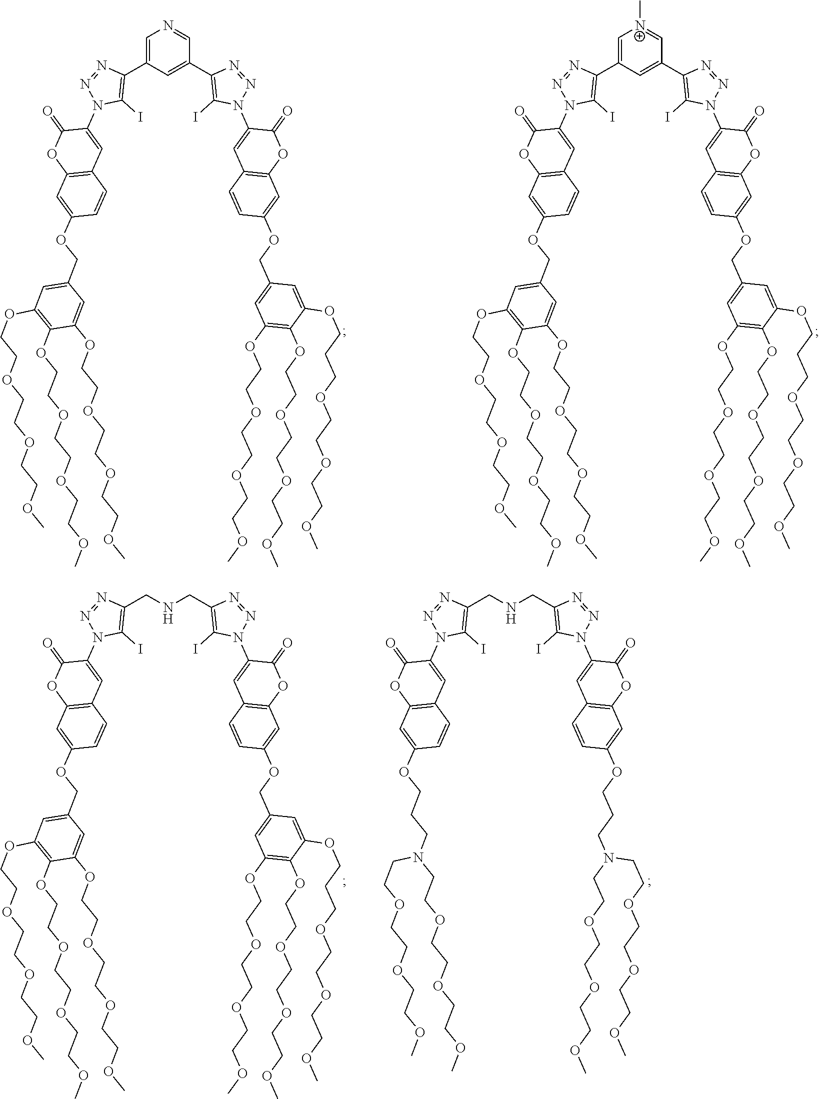

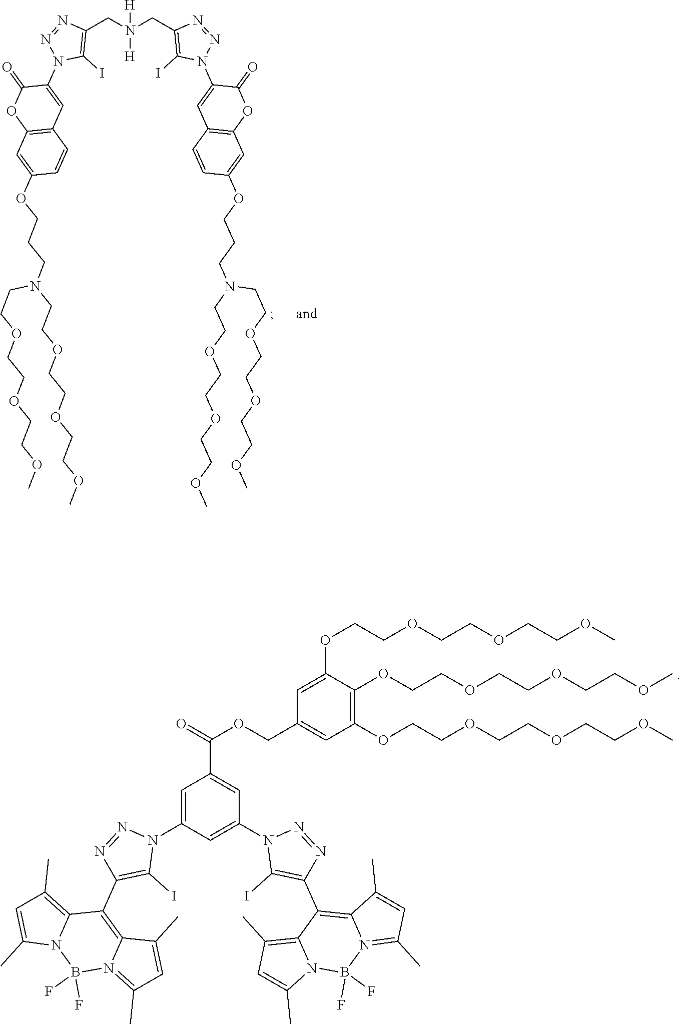

Compounds of Formula I (and any sub-formula thereof) may exist in protonated form and/or as a salt. It will be understood that such protonated and salt forms are within the scope of the invention.

The compounds of the present invention may be bound to a solid support. It will be understood that any structural modifications to the compound of Formula I that occur when it is bound to such a solid support are within the scope of the invention. In an embodiment, the solid support is glass.

### Sensors of the Invention

As described hereinbefore, in accordance with the second aspect of the present invention there is provided a sensor comprising a compound having a structure according to Formula I (or any associated sub-definitions of Formula I) defined hereinbefore.

### Uses of the Invention

As described hereinbefore, in accordance with the third aspect of the present invention there is provided a use of a compound according to the first aspect of the invention or a sensor according to the second aspect of the invention for detecting the presence or absence of sulphide in a solution.

In an embodiment, the solution is an aqueous solution. Suitably, the aqueous solution comprises >50% v/v water. More suitably, the aqueous solution comprises >70% v/v water. Even more suitably, the aqueous solution comprises >90% v/v water. Most suitably, the aqueous solution comprises >95% v/v water.

In an embodiment, the solution is wastewater or sewage. Sulphide may be detected as part of a wastewater treatment process.

In an embodiment, the aqueous sample is a biological sample. In such embodiments, detecting the presence of sulphide in the biological sample may be conducted in an in vitro or an in vivo manner.

### Methods of the Invention

As described hereinbefore, in accordance with the fourth aspect of the present invention there is provided a method of detecting the presence or absence of sulphide in a solution, the method comprising:
a) contacting a compound according to the first aspect of the invention or a sensor according to the second aspect of the invention with the solution; and
b) detecting the presence or absence of sulphide in the solution.

In an embodiment, the solution is an aqueous solution. Suitably, the aqueous solution comprises >50% v/v water. More suitably, the aqueous solution comprises >70% v/v water. Even more suitably, the aqueous solution comprises >90% v/v water. Most suitably, the aqueous solution comprises >95% v/v water.

In an embodiment, the solution is wastewater or sewage. Detecting the presence or absence of sulphide may be conducted as part of a wastewater treatment process.

In an embodiment, the solution is a biological sample. In such embodiments, the method may be conducted in vitro or in vivo.

Suitably, when present in the solution, sulphide exists as HS⁻.

Step b) may comprise detecting the presence or absence of sulphide in the aqueous sample by monitoring the fluorescence of a compound having a structure according Formula I defined hereinbefore.

Suitably, the method defined herein is performed in a continuous manner.

The following numbered statements 1 to 95 are not claims, but instead describe particular aspects and embodiments of the invention:

1. A compound having a structure according to Formula I, shown below:

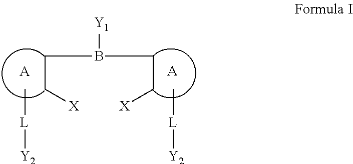

wherein
each X is independently I or Br;
each ring A is independently:
   i) an electron deficient 5- or 6-membered nitrogen-containing heteroaryl that is optionally substituted with one or more groups Rₑ, where each Rₑ is independently selected from (1-6C)alkyl, oxygen and aryl;
   or
   ii) a phenyl that is substituted with one or more groups R_{f}, where each R_{f} is an electron withdrawing group;
each L is independently absent (such that ring A is directly bound to Y₂) or a π-conjugated linker that separates ring A from Y₂ by a distance of 4 atoms or fewer;
Y₁ is absent or is a fluorophore comprising coumarin or BODIPY;
Y₂ is absent or is a fluorophore comprising coumarin or BODIPY;
B is a backbone group of the following structure:

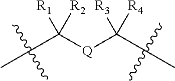

wherein [symbol] denotes the point of attachment to each ring A; and
   i) R₁, R₂, R₃ and R₄ are independently selected from hydrogen, halo, hydroxy, (1-4C)alkyl, (2-4C)alkenyl, (2-4C)alkynyl, (1-4C)haloalkyl and (1-4C)alkoxy; and Q is selected from CRₐR_{b}, N⁺RₐR_{b} and NR_{c}, where Rₐ, R_{b} and R_{c} are each independently hydrogen, (1-6C)alkyl or aryl;
   or
   ii) R₁ and R₄ are absent, and R₂ and R₃ are linked to one another, such that when taken in combination with the atoms to which they are attached, they form a 5- or 6-membered heteroaryl or an aryl that is optionally substituted with one or more groups Rₐ, where each Rₐ is independently selected from halo, hydroxy, nitro, cyano, (1-6C)alkyl, (2-6C)alkenyl, (2-6C)alkynyl, (1-6C)haloalkyl, (1-6C)alkoxy, —NR_{d1}R_{d2}, —C(O)—R_{d1}, and —C(O)—OR_{d1}, where R_{d1} and R_{d2} are each independently selected from hydrogen and (1-4C)alkyl; and
   Q is an atom in the 5- or 6-membered heteroaryl ring or the aryl ring;
with the proviso that Y₁ and Y₂ are not both absent; and
wherein the compound of Formula I is substituted with one or more water solubilising groups, Z.

2. The compound according to statement 1, wherein each X is I.

3. The compound according to statement 1 or 2, wherein each ring A is independently an electron deficient 5-membered nitrogen-containing heteroaryl that is optionally substituted with one or more groups Rₑ.

4. The compound according to statement 1, 2 or 3, wherein each ring A is independently an electron deficient 5-membered nitrogen-containing heteroaryl comprising 1, 2 or 3 heteroatoms that is optionally substituted with one or more groups Rₑ.

5. The compound according to any one of the preceding statements, wherein each ring A is independently an electron deficient 5-membered nitrogen-containing heteroaryl comprising 1, 2 or 3 nitrogen atoms that is optionally substituted with one or more groups Rₑ.

6. The compound according to any one of the preceding statements, wherein each ring A is independently an electron deficient 5-membered nitrogen-containing heteroaryl comprising 2 or 3 nitrogen atoms that is optionally substituted with one or more groups Rₑ.

7. The compound according to any one of the preceding statements, wherein each ring A is independently an electron deficient 5-membered nitrogen-containing heteroaryl comprising 3 nitrogen atoms that is optionally substituted with one or more groups Rₑ.

8. The compound according to any one of the preceding statements, wherein each ring A is independently a triazole or an oxadiazole that is optionally substituted with one or more groups Rₑ.

9. The compound according to any one of the preceding statements, wherein each ring A is independently a triazole that is optionally substituted with one or more groups Rₑ.

10. The compound according to any one of the preceding statements, wherein each Rₑ is independently selected from (1-4C)alkyl, oxygen and aryl.

11. The compound according to any one of the preceding statements, wherein each Rₑ is independently selected from (1-3C)alkyl, oxygen and 6-membered aryl.

12. The compound according to any one of the preceding statements, wherein each Rₑ is independently selected from methyl, oxygen and phenyl.

13. The compound according to any one of the preceding statements, wherein each ring A is independently a phenyl that is substituted with one or more groups R_{f}, where each R_{f} is an electron withdrawing group.

14. The compound according to any one of the preceding statements, wherein each ring A is independently a phenyl that is substituted with 1, 2 or 3 groups R_{f}, where each R_{f} is an electron withdrawing group.

15. The compound according to any one of the preceding statements, wherein each ring A is independently a phenyl that is substituted with one or two groups R_{f}, where each R_{f} is an electron withdrawing group.

16. The compound according to any one of the preceding statements, wherein each ring A is independently a phenyl that is substituted with one group R_{f}, where each R_{f} is an electron withdrawing group.

17. The compound according to any one of the preceding statements, wherein each R_{f} is independently selected from fluoro, chloro, nitro, cyano, (1-3C)haloalkyl, —C(O)—R_{f1} and —C(O)O—R_{f1}, where R_{f1} is independently selected from hydrogen and (1-2C)alkyl.

18. The compound according to any one of the preceding statements, wherein each R_{f} is independently selected from fluoro, nitro, cyano, —C(O)—R_{f1} and —C(O)O—R_{f1}, where R_{f1} is independently selected from hydrogen and (1-2C)alkyl (e.g. methyl).

19. The compound according to any one of the preceding statements, wherein each R_{f} is independently selected from fluoro, chloro, nitro and cyano.

20. The compound according to any one of the preceding statements, wherein each L is independently absent (such that ring A is directly bound to Y₂) or a π-conjugated linker that separates ring A from Y₂ by a distance of 2-4 atoms.

21. The compound according to any one of the preceding statements, wherein each L is independently absent or a π-conjugated linker that separates ring A from Y₂ by a distance of 4 atoms or less, wherein the π-conjugated linker comprises fewer than 20 atoms in total.

22. The compound according to any one of the preceding statements, wherein each L is independently absent or a T-conjugated linker that separates ring A from Y₂ by a distance of 4 atoms or less, wherein the π-conjugated linker comprises fewer than 10 atoms in total.

23. The compound according to any one of the preceding statements, wherein each L is absent (such that ring A is directly bound to Y₂).

24. The compound according to any one of the preceding statements, wherein Y₁ is absent or is a fluorophore comprising coumarin or BODIPY.

25. The compound according to any one of the preceding statements, wherein Y₁ is absent.

26. The compound according to any one of the preceding statements, wherein Y₂ is absent or is a fluorophore comprising coumarin or BODIPY.

27. The compound according to any one of the preceding statements, wherein Y₂ is a fluorophore comprising coumarin or BODIPY.

28. The compound according to any one of the preceding statements, wherein Y₂ is a fluorophore selected from:

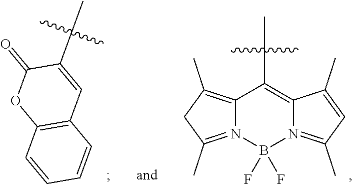

wherein [symbol] denotes the point of attachment to L (or ring A when L is absent).

29. The compound according to any one of the preceding statements, wherein Y₁ and Y₂ are not both absent and Y₁ and Y₂ are not both a fluorophore comprising coumarin or BODIPY.

30. The compound according to any one of the preceding statements, wherein Y₁ is absent and Y₂ is a fluorophore comprising coumarin or BODIPY.

31. The compound according to any one of the preceding statements, wherein R₁, R₂, R₃ and R₄ are independently selected from hydrogen, chloro, fluoro, hydroxy, (1-3C)alkyl, (2-3C)alkenyl, (2-3C)alkynyl, (1-3C)haloalkyl and (1-3C)alkoxy.

32. The compound according to any one of the preceding statements, wherein R₁, R₂, R₃ and R₄ are independently selected from hydrogen, (1-3C)alkyl, (1-3C)haloalkyl and (1-3C)alkoxy.

33. The compound according to any one of the preceding statements, wherein R₁, R₂, R₃ and R₄ are independently selected from hydrogen and (1-3C)alkyl.

34. The compound according to any one of the preceding statements, wherein R₁, R₂, R₃ and R₄ are independently selected from hydrogen.

35. The compound according to any one of the preceding statements, wherein Q is selected from CRₐR_{b}, N⁺RₐR_{b} and NR_{c}, where Rₐ, R_{b} and R_{c} are each independently hydrogen, (1-3C)alkyl or phenyl.

36. The compound according to any one of the preceding statements, wherein Q is selected from N⁺RₐR_{b} and NR_{c}, where Rₐ, R_{b} and R_{c} are each independently hydrogen or methyl.

37. The compound according to any one of the preceding statements, wherein Q is selected from N⁺RₐR_{b} and NR_{c}, where Rₐ, R_{b} and R_{c} are each independently hydrogen.

38. The compound according to any one of the preceding statements, wherein Q is NR_{c}, where R_{c} is hydrogen.

39. The compound according to any one of statements 1 to 37, wherein B is a backbone group having a structure selected from the following:

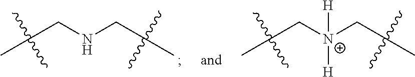

wherein [symbol] denotes the point of attachment to each ring A.

40. The compound according to any one of statements 1-30, wherein R₁ and R₄ are absent, and R₂ and R₃ are linked to one another, such that when taken in combination with the atoms to which they are attached, they form a 5- or 6-membered heteroaryl or a phenyl that is optionally substituted with one or more groups R_{d}.

41. The compound according to statement 40, wherein R₁ and R₄ are absent, and R₂ and R₃ are linked to one another, such that when taken in combination with the atoms to which they are attached, they form a 6-membered nitrogen-containing heteroaryl or a phenyl that is optionally substituted with one or more groups R_{d}.

42 The compound according to statement 40, wherein R₁ and R₄ are absent, and R₂ and R₃ are linked to one another, such that when taken in combination with the atoms to which they are attached, they form a 6-membered heteroaryl containing 1 or 2 nitrogen atoms or a phenyl that is optionally substituted with one or more groups R_{d}.

43. The compound according to statement 40, wherein R₁ and R₄ are absent, and R₂ and R₃ are linked to one another, such that when taken in combination with the atoms to which they are attached, they form a pyrimidine, a pyridine or a phenyl that is optionally substituted with one or more groups R_{d}.

44. The compound according to any one of statements 40-43, wherein each Rₐ is independently selected from halo, hydroxy, nitro, cyano, (1-3C)alkyl, (2-3C)alkenyl, (2-3C)alkynyl, (1-3C)haloalkyl, (1-3C)alkoxy, —NR_{d1}R_{d2}, —C(O)—R_{d1}, and —C(O)—OR_{d1}, where R_{d1} and R_{d2} are each independently selected from hydrogen and (1-4C)alkyl.

45. The compound according to any one of statements 40-44, wherein each R_{d} is independently selected from nitro, (1-3C)alkyl, —C(O)—R_{d1}, and —C(O)—OR_{d1}, where R_{d1} is selected from hydrogen and (1-2C)alkyl.

46. The compound according to any one of statements 40-45, wherein each Rₐ is independently selected from nitro, (1-2C)alkyl (e.g. methyl), and —C(O)—R_{d1}, where R_{d1} is hydrogen.

47. The compound according to any one of statements 40-46, wherein each R_{d} is independently selected from (1-2C)alkyl (e.g. methyl).

48. The compound according to any one of statements 40-47, wherein Q is a ring atom within the 5- or 6-membered heteroaryl ring or the aryl ring selected from a carbon atom and a nitrogen atom.

49. The compound according to any one of statements 40-47, wherein R₁ and R₄ are absent, and R₂ and R₃ are linked to one another, such that when taken in combination with the atoms to which they are attached, they form one of the following structures:

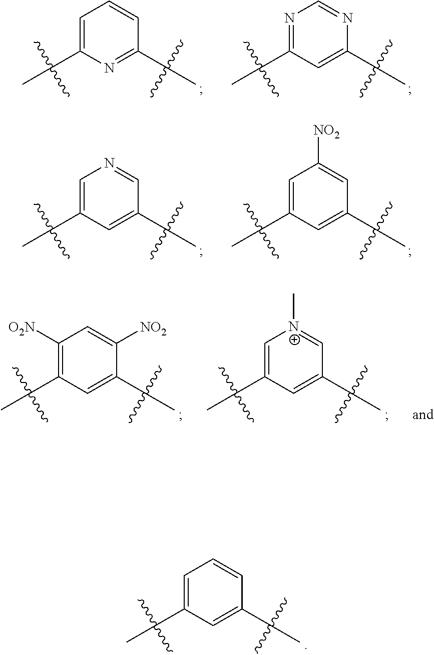

wherein [symbol] denotes the point of attachment to each ring A.

50. The compound according to any one of statements 40-48, wherein R₁ and R₄ are absent, and R₂ and R₃ are linked to one another, such that when taken in combination with the atoms to which they are attached, they form one of the following structures:

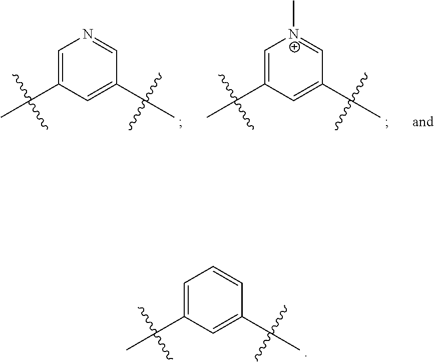

wherein [symbol] denotes the point of attachment to each ring A.

51. The compound according to any one of the preceding statements, wherein the one or more water solubilising groups, Z, comprises i) one or more poly(2-4C)alkylene glycol groups, each comprising 2-10 repeating (2-4C)alkylene units, or ii) one or more carboxylate or carboxylate salt groups.

52. The compound according to any one of the preceding statements, wherein the one or more water solubilising groups, Z, comprises i) one or more poly(2-3C)alkylene glycol groups, each comprising 2-8 repeating (2-3C)alkylene units, or ii) two or more carboxylate or carboxylate salt groups.

53. The compound according to any one of the preceding statements, wherein the one or more water solubilising groups, Z, comprises i) two or more polyethylene glycol groups, each comprising 3-6 repeating ethylene glycol units, or ii) three or more carboxylate or carboxylate salt groups.

54. The compound according to any one of statements 1 to 50, wherein the one or more water solubilising groups, Z, is independently selected from one of the following structures:

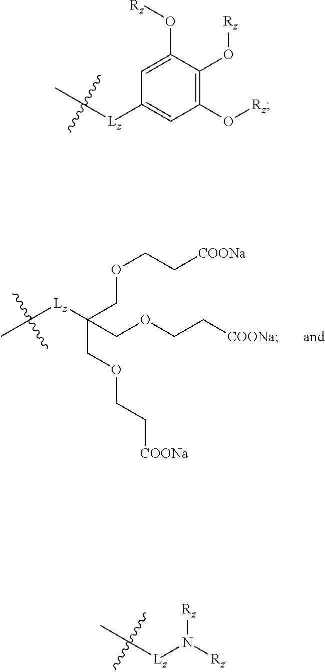

wherein [symbol] denotes the point of attachment of Z within the compound of Formula I (e.g. the point of attachment to Y² or B);
L_{Z} is absent or is a linking group containing 15 atoms or fewer; and
R_{z} is a polyethylene glycol group, each comprising 2-10 (or 2-8 or 3-6) repeating units.

55. The compound according to statement 50, wherein L_{Z} is absent or is a linking group -(L_{Z1})-(L_{Z2})-(L_{Z3})—, where L_{Z1} and L_{Z3} are independently absent or a (1-4C)alkylene or (2-4C)alkenylene group, and L_{Z2} is absent or is a group —O—, —NH—, —OC(O)—, —C(O)O—, —C(O)—, —NHC(O)— or —C(O) NH—.

56. The compound according to any one of the preceding statements, wherein the compound of Formula I has a structure according to Formula Ia (which is a sub-definition of Formula I), shown below:

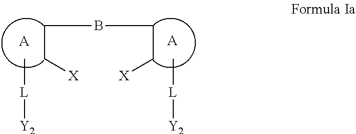

wherein
the compound of Formula Ia is substituted with one or more water solubilising groups, Z; and
Y₂, B, Z, A, L and X, and any associated sub-groups, have any of the definitions outlined in any one of the preceding statements.

57. The compound according to any one of statements 1-55, wherein the compound of Formula I has a structure according to Formula Ib (which is a sub-definition of Formula I), shown below:

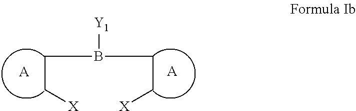

wherein
the compound of Formula Ib is substituted with one or more water solubilising groups, Z; and
Y₁, B, Z, A and X, and any associated sub-groups, have any of the definitions outlined in any one of statements 1-54.

58. The compound according to any one of statements 1-55, wherein the compound of Formula I has a structure according to Formula II (which is a sub-definition of Formula I), shown below:

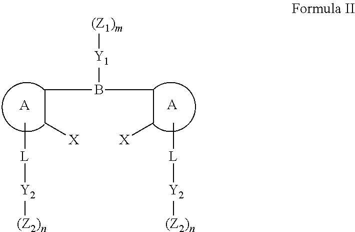

wherein
B, A, L, X, Y₁ and Y₂, and any associated sub-groups, have any of the definitions outlined in any one of statements 1-55;
Z₁ and Z₂ are each independently a water solubilising group having any of the definitions outlined in statements 51-55 in relation to Z;
m is 0, 1 or 2; and
n is 0, 1 or 2;
with the proviso that m and n are not both 0.

59. The compound according to statement 58, wherein m is 0 or 1.

60. The compound according to statement 58 or 59, wherein n is 0 or 1.

61. The compound according to statement 58, 59 or 60, wherein m is 0 and n is 1.

62. The compound according to any one of statements 58-61, wherein
m is 0 and n is 1; and
Y¹ is absent and Y₂ is a fluorophore:

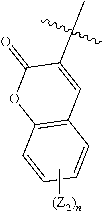

wherein [symbol] denotes the point of attachment to L (or ring A when L is absent).

63 The compound according to any one of statements 58-62, wherein
m is 0 and n is 1; and
Y¹ is absent and Y₂ is a fluorophore:

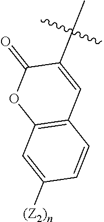

wherein [symbol] denotes the point of attachment to L (or ring A when L is absent).

64. The compound according to statement 58, wherein
m is 1 and n is 0; and
Y₁ is absent and Y₂ is a fluorophore:

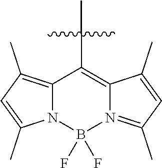

wherein [symbol] denotes the point of attachment to L (or ring A when L is absent).

65. The compound according to any one of statements 1-55 and 58-64, wherein the compound of Formula I has a structure according to Formula IIa (which is a sub-definition of Formula II), shown below:

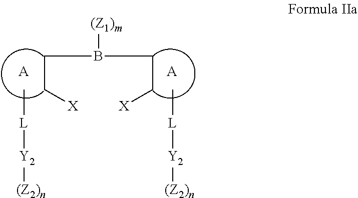

wherein
Y₂, m, n, Z₁, Z₂, B, A, L and X, and any associated sub-groups, have any of the definitions outlined in any one of statements 1-55 and 58-64.

66. The compound according to any one of statements 1-55 and 58-65, wherein the compound of Formula I has a structure according to Formula IIa-i, IIa-ii, IIa-iii or IIa-iv (which are sub-definitions of Formula IIa), shown below:

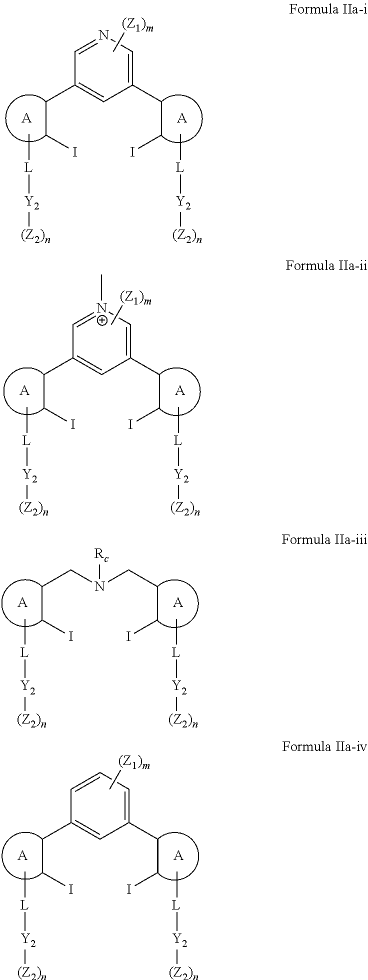

wherein Y₂, m, n, R_{c}, Z₁, Z₂, A and L, and any associated sub-groups, have any of the definitions outlined in any one of statements 1-55 and 58-65.

67. The compound according to any one of statements 1-55 and 58-66, wherein the compound of Formula I has a structure according to Formula IIa-v (which is a sub-definition of Formula IIa), shown below:

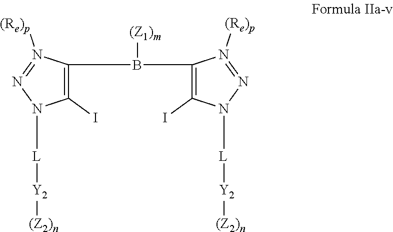

wherein
p is 0 or 1; and
Y₂, m, n, Rₑ, Z₁, Z₂, B and L, and any associated sub-groups, have any of the definitions outlined in any one of statements 1-55 and 58-66.

68 The compound according to any one of statements 1-55 and 58-66, wherein the compound of Formula I has a structure according to Formula IIa-vi (which is a sub-definition of Formula IIa), shown below:

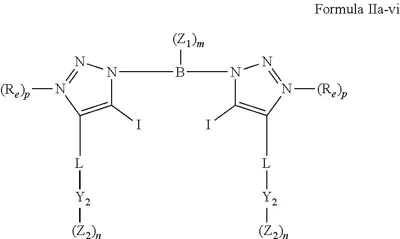

wherein
p is 0 or 1; and
Y₂, m, n, Rₑ, Z₁, Z₂, B and each L, and any associated sub-groups, have any of the definitions outlined in any one of statements 1-55 and 58-67.

69. The compound according to any one of statements 1-55 and 58-64, wherein the compound of Formula I has a structure according to Formula IIb (which is a sub-definition of Formula II), shown below:

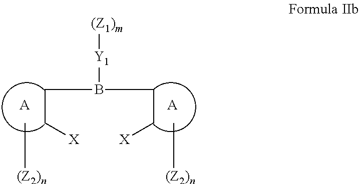

wherein
Y₁, m, n, Z₁, Z₂, B, A and X, and any associated sub-groups, have any of the definitions outlined in any one of statements 1-55 and 58-64.

70. The compound according to any one of statements 1-55, 58-64 and 69, wherein the compound of Formula I has a structure according to Formula IIb-i or IIb-ii (which are sub-definitions of Formula IIb), shown below:

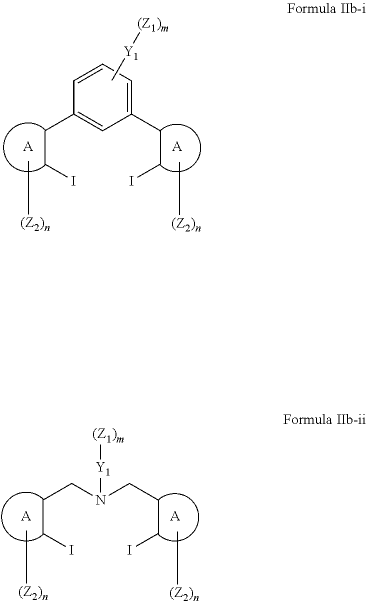

wherein
Y₁, m, n, Z₁, Z₂, B, A and X, and any associated sub-groups, have any of the definitions outlined in any one of statements 1-55, 58-64 and 69.

71. The compound according to statement 1, wherein the compound is selected from any one of the following:

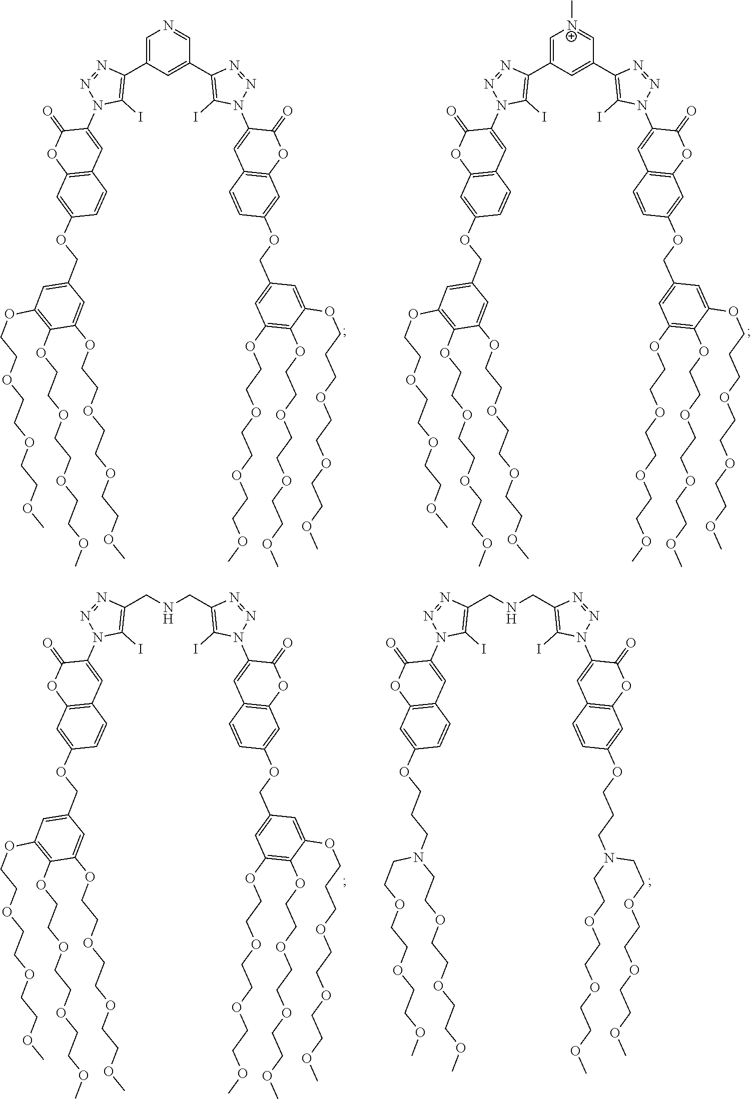

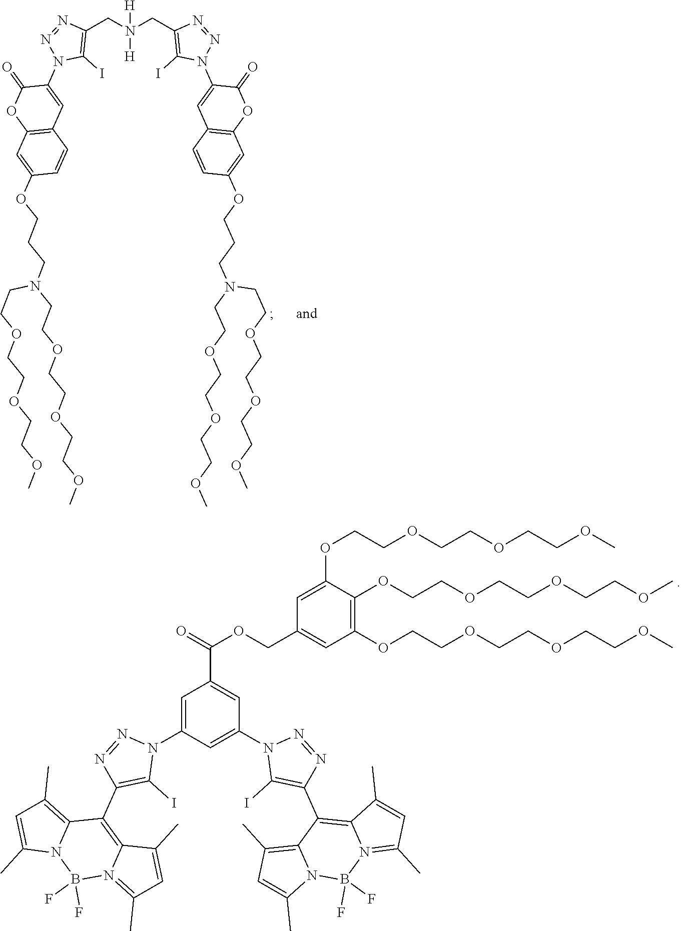

72. The compound according to any one of the preceding statements, wherein the compound is bound to a solid support.

73. The compound of statement 72, wherein the solid support is glass.

74. A sensor comprising a compound according to any one of statements 1 to 73.

75. Use of a compound according to any one of statements 1 to 73 or a sensor according to statement 74 for detecting the presence or absence of sulphide in a solution.

76. The use according to statement 75, wherein the solution is an aqueous solution.

77. The use according to statement 76, wherein the aqueous solution comprises >50% v/v water.

78. The use according to statement 76, wherein the aqueous solution comprises >70% v/v water.

79. The use according to statement 76, wherein the aqueous solution comprises >90% v/v water.

80. The use according to statement 76, wherein the aqueous solution comprises >95% v/v water.

81. The use according to any one of statements 75 to 80, wherein the solution is wastewater or sewage.

82. The use according to any one of statements 75 to 80, wherein the solution is a biological sample.

83. The use according to statement 82, wherein detecting the presence or absence of sulphide in the biological sample is performed in vitro or in vivo.

84. A method of detecting the presence or absence of sulphide in a solution, the method comprising:
a) contacting a compound according to any one of statements 1 to 73 or a sensor according to statement 74 with the solution; and
b) detecting the presence or absence of sulphide in the solution.

85. The method according to statement 84, wherein the solution is an aqueous solution.

86. The method according to statement 85, wherein the aqueous solution comprises >50% v/v water.

87. The method according to statement 85, wherein the aqueous solution comprises >70% v/v water.

88. The method according to statement 85, wherein the aqueous solution comprises >90% v/v water.

89. The method according to statement 85, wherein the aqueous solution comprises >95% v/v water.

90. The method according to any one of statements 84 to 89, wherein the solution is wastewater or sewage.

91. The method according to any one of statements 84 to 89, wherein the solution is a biological sample.

92. The method according to statement 91, wherein the method is performed in vitro or in vivo.

93. The method according to any one of statements 84 to 92, wherein, when present in the solution, sulphide exists as HS⁻.

94. The method according to any one of statements 84 to 93, wherein step b) comprises detecting the presence or absence of sulphide in the solution by monitoring the fluorescence of the compound according to any one of statements 1 to 73 or the sensor according to statement 74.

95. The method according to any one of statements 84 to 94, wherein the method is performed in a continuous manner.

### EXAMPLES

One or more examples of the invention will now be described, for the purpose of illustration only, with reference to the accompanying figures:

FIG. 1. Fluorescence emission change of 10 μM XB1 on addition of NaHS in pH 7.4 10 mM aqueous HEPES buffer solution. The arrow on the spectrum denotes the direction of change in fluorescence intensity (decrease) upon anion addition.

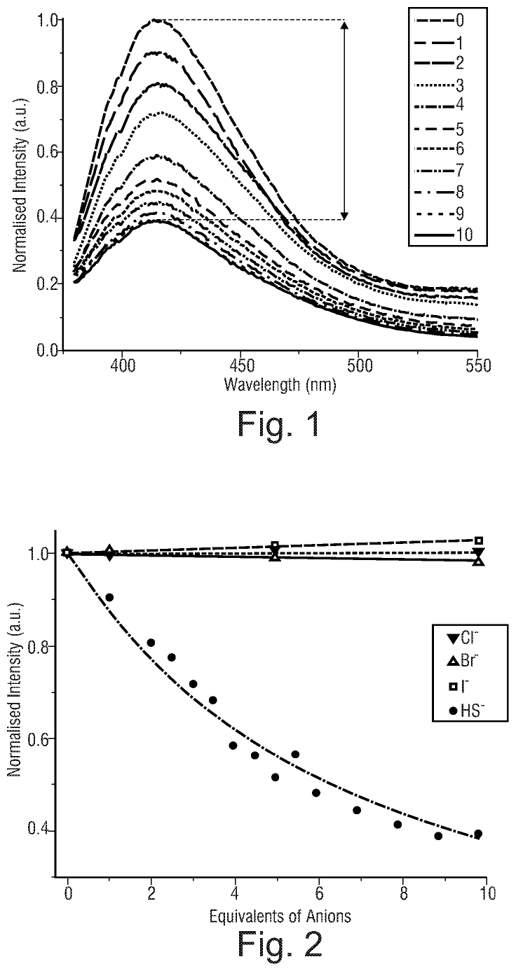

FIG. 2. Binding isotherms and Bindfit analysis^{23,24} fit of fluorescence titration data monitoring λₘₐₓ (400-420 nm) of XB1 upon addition of NaCl, NaBr, NaI and NaHS in pH 7.4 10 mM aqueous HEPES buffer solution.

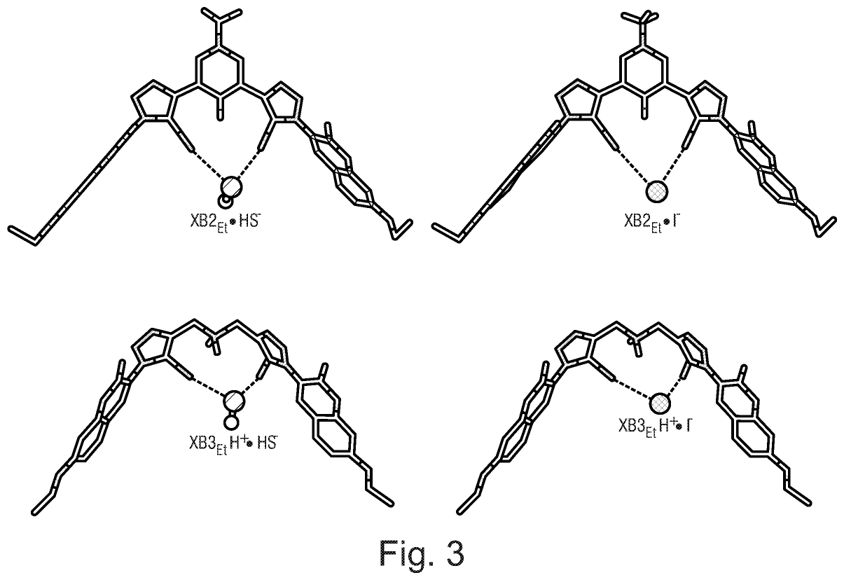

FIG. 3. DFT PBE0-D3 optimised structures of XB2_{Et} (top) and XB3_{Et}H⁺ (bottom) model receptors halogen bonded to HS⁻ (left) and I⁻ (right) anions. The XB interactions are sketched as purple dashed lines.

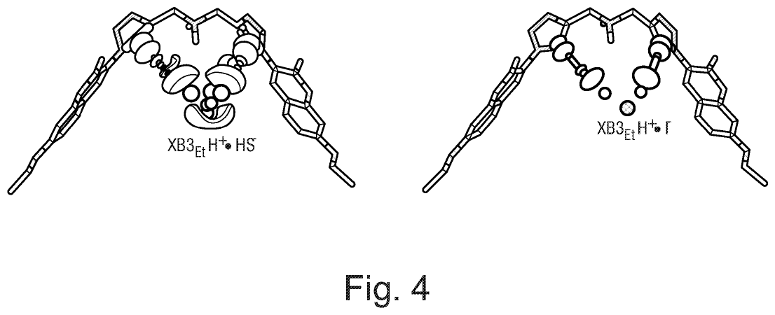

FIG. 4. Electron density difference maps for the XB3_{Et}H⁺ associations with HS⁻ (left) and I⁻ (right) highlighting the superior electron and charge transfer in the HS⁻ host-guest association.

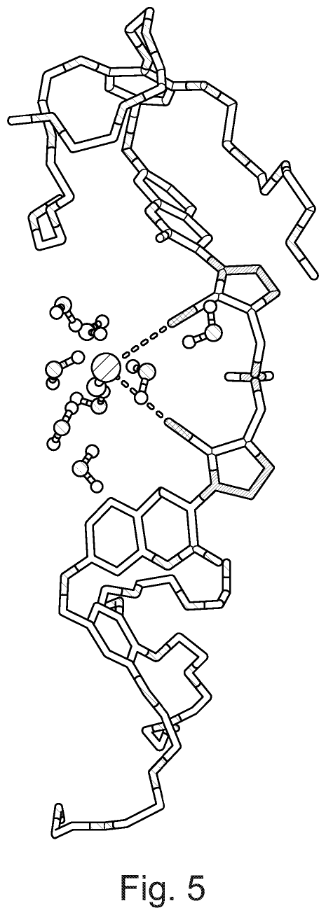

FIG. 5. A snapshot of a MD simulation of HS⁻ associated with XB3H⁺ showing the anion enclosed by water molecules and the random orientation of the TEG flexible chains. Most hydrogen atoms were hidden for clarity.

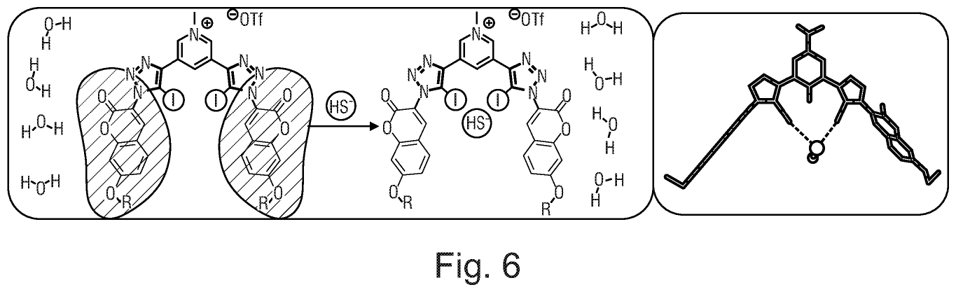

FIG. 6. Simulation of the effect on fluorescence emission when HS⁻ is associated with XB2 in water. Also shown is DFT PBE0-D3 optimised structures of XB2 model receptors halogen bonded to the HS⁻ anion. The XB interactions are sketched as purple dashed lines.

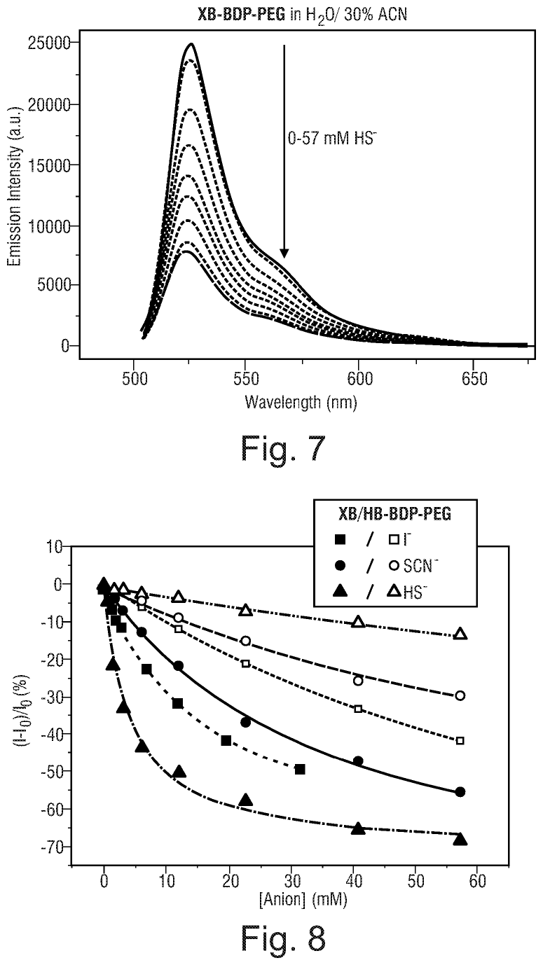

FIG. 7. Fluorescence emission intensity change of XB-BDP-PEG in H₂O/30% ACN upon titration with HS⁻.

FIG. 8. Relative fluorescence emission decrease of XB-BDP-PEG (filled symbols) and HB-BDP-PEG (empty symbols) in H₂O/30% ACN upon titration with various anions.

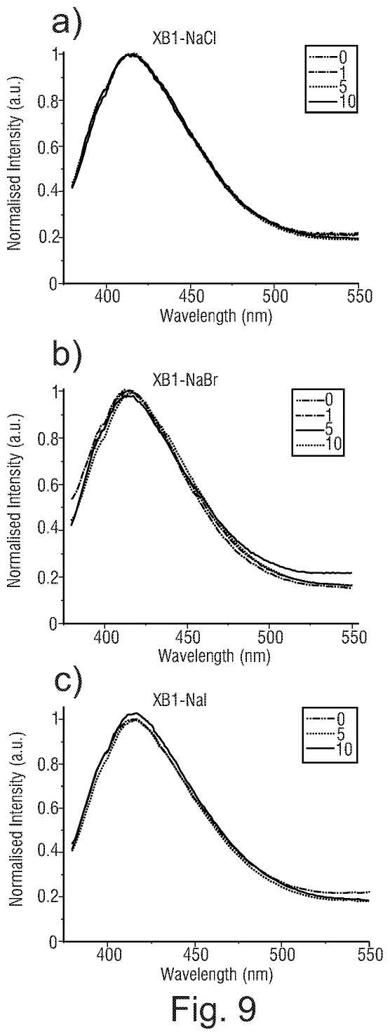

FIG. 9. Fluorescence emission change of 10 μM XB1 on addition of a) NaCl b) NaBr, and c) NaI in pH 7.4 10 mM aqueous HEPES buffer solution.

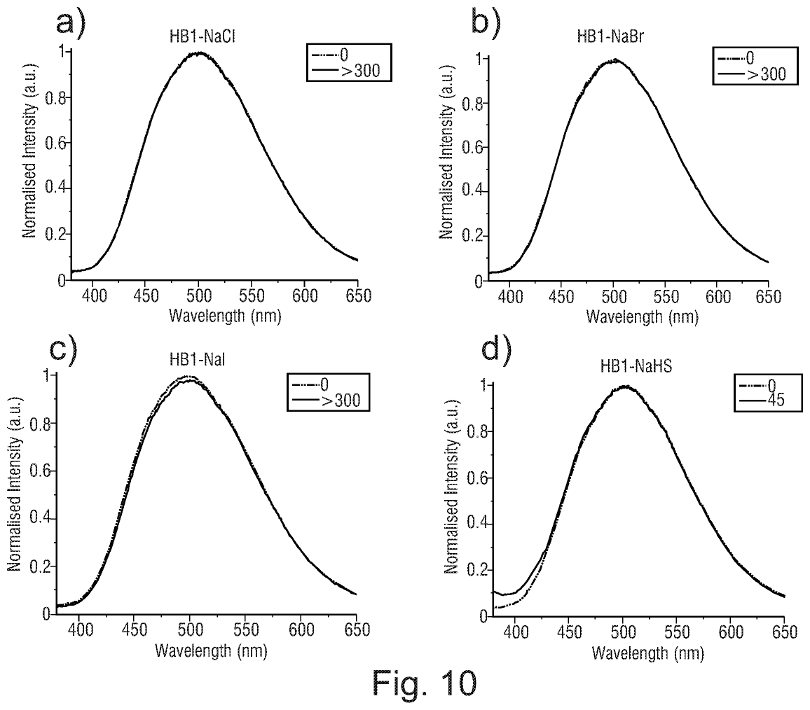

FIG. 10. Fluorescence emission change of 10 μM HB1 on addition of a) NaCl b) NaBr, c) NaI d) NaHS in pH 7.4 10 mM aqueous HEPES buffer solution.

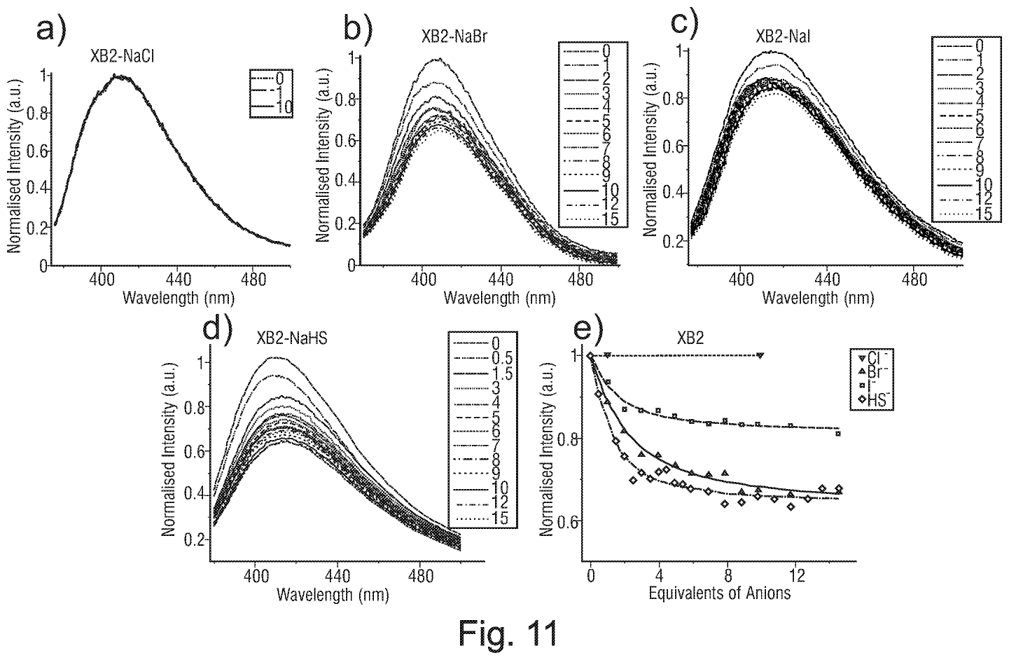

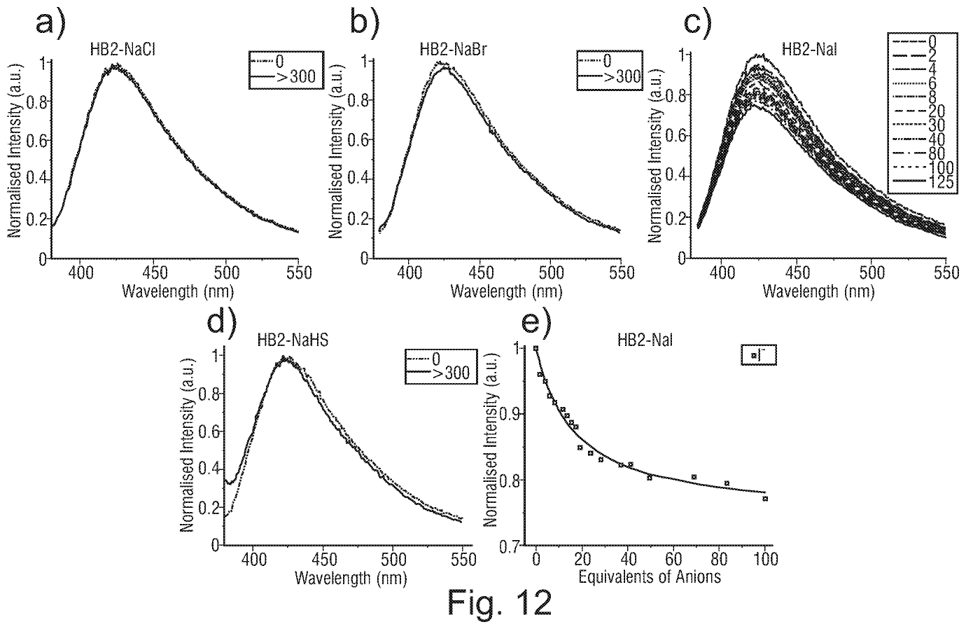

FIG. 11. Fluorescence emission change of 10 μM XB2 on addition of a) NaCl b) NaBr, c) NaI d) NaHS in pH 7.4 10 mM aqueous HEPES buffer solution e) BindFit analysis fit of fluorescence titration data monitoring λₘₐₓ (400-420 nm) of receptor XB2 upon addition of NaCl, NaBr, NaI and NaHS in pH 7.4 10 mM aqueous HEPES buffer solution.

FIG. 12. Fluorescence emission change of 10 μM HB2 on addition of a) NaCl b) NaBr, c) NaI d) NaHS in pH 7.4 10 mM aqueous HEPES buffer solution e) BindFit analysis fit of fluorescence titration data monitoring λₘₐₓ (400-420 nm) of receptor HB2 upon addition of NaI in pH 7.4 10 mM aqueous HEPES buffer solution.

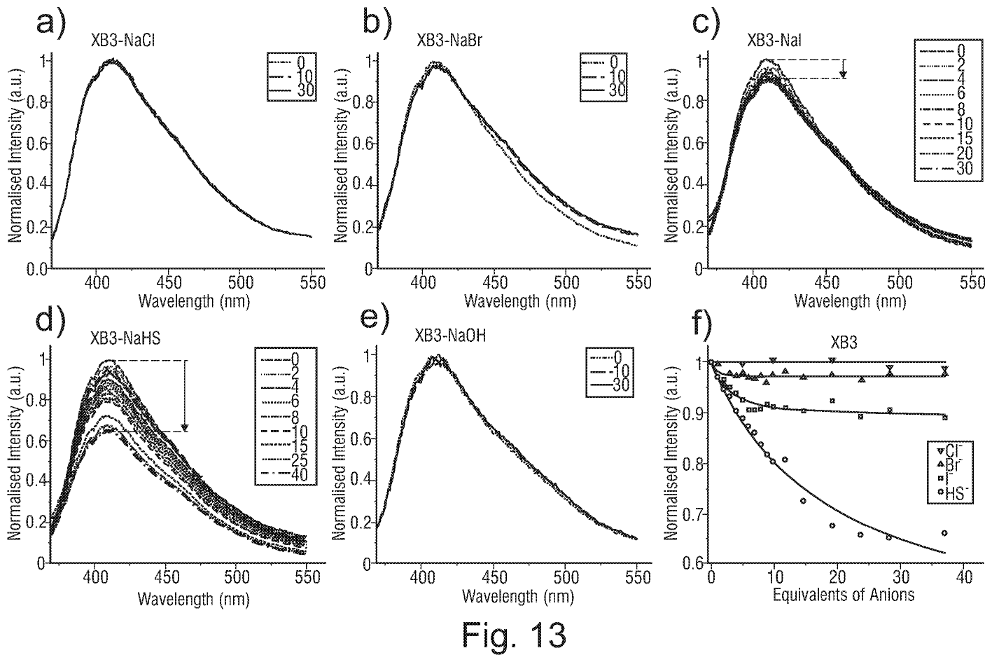

FIG. 13. Fluorescence emission change of 10 μM XB3 on addition of a) NaCl b) NaBr, c) NaI d) NaHS e) NaOH in pH 7.4 10 mM aqueous HEPES buffer solution e) BindFit analysis fit of fluorescence titration data monitoring λₘₐₓ (400-420 nm) of receptor XB3 upon addition of NaCl, NaBr, NaI and NaHS in pH 7.4 10 mM aqueous HEPES buffer solution.

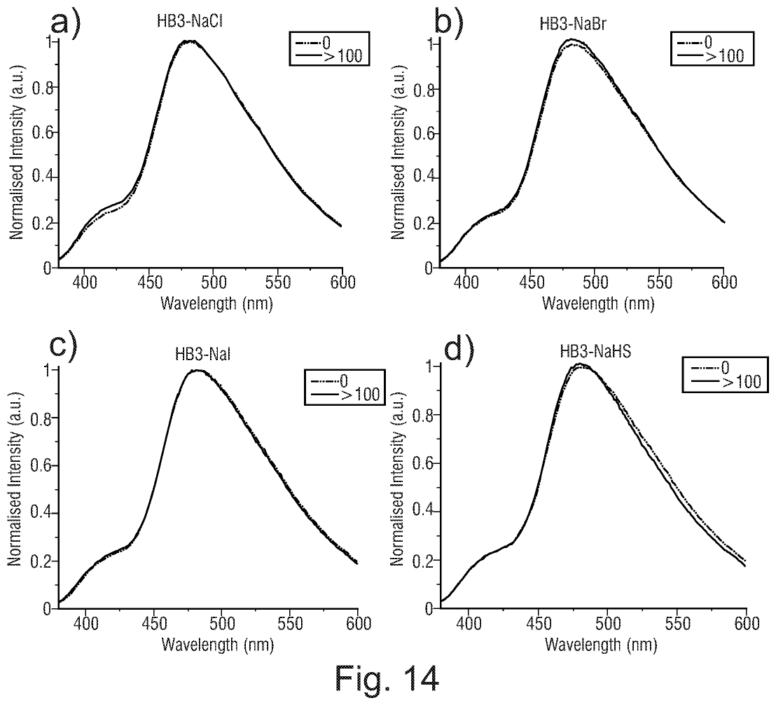

FIG. 14. Fluorescence emission change of 10 μM HB3 on addition of a) NaCl b) NaBr, c) NaI d) NaHS in pH 7.4 10 mM aqueous HEPES buffer solution.

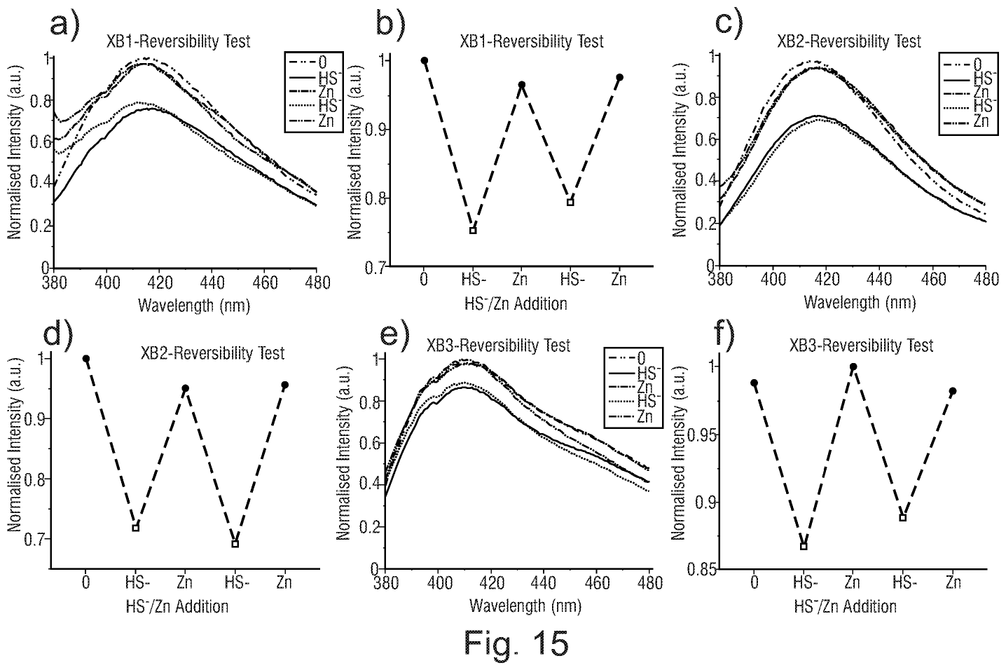

FIG. 15. Recorded emission spectra of a) XB1 c) XB2 e) XB3 upon addition of 5 equivalents of NaHS and Zn(OTf)₂, repeated for two cycles. Plotted averaged peak normalised intensity (400-420 nm) with each cycle for b) XB1 d) XB2 f) XB3.

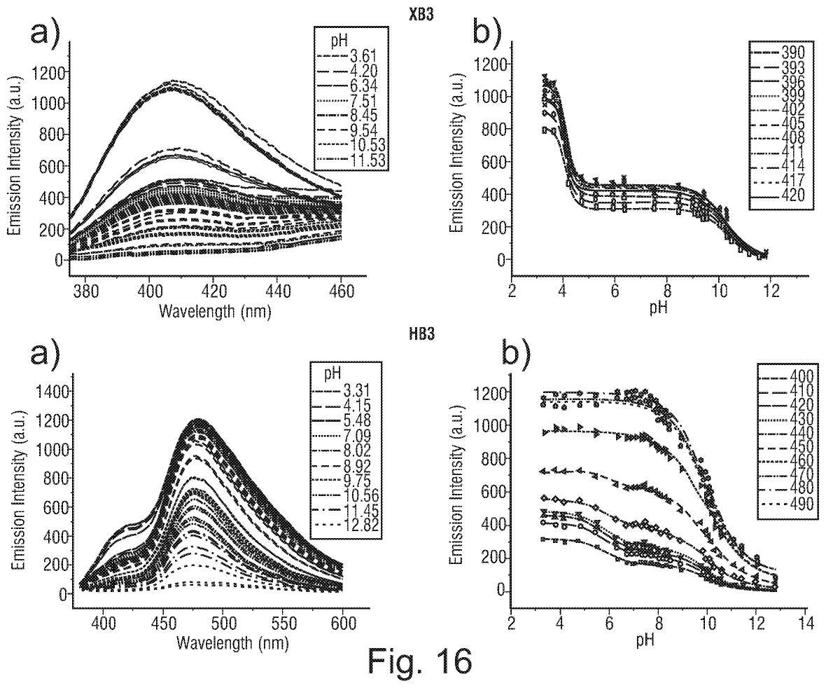

FIG. 16. XB3: a) XB3 emission intensity change with decreasing pH; b) Emission intensity change for select wavelengths with changing pH; pKa₁=4.07 (±0.01), pKa₂=10.29 (±0.01). HB3: a) HB3 emission intensity change with decreasing pH; b) Emission intensity change for select wavelengths with changing pH; pKa₁=5.91 (±0.07), pKa₂=9.98 (±0.01).

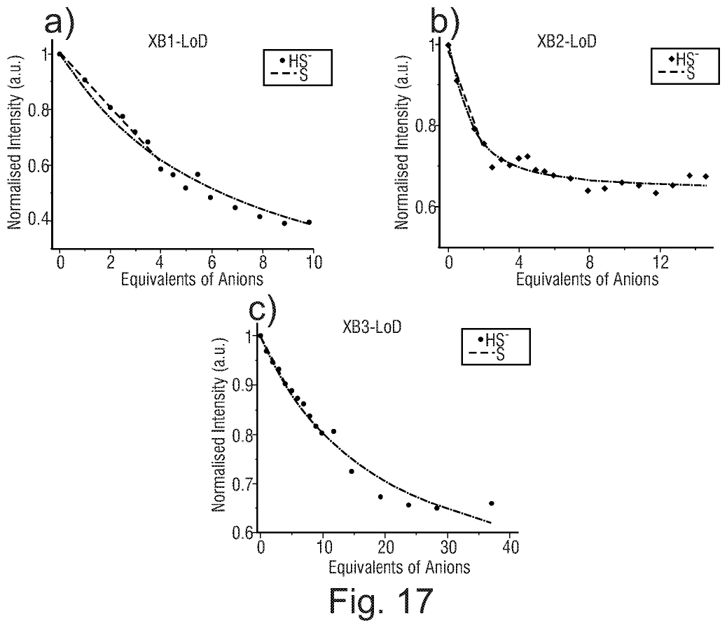

FIG. 17. BindFit analysis fit of fluorescence titration data monitoring λₘₐₓ (400-420 nm) of receptors a) XB1 b) XB2 c) XB3 upon addition of NaHS in pH 7.4 10 mM aqueous HEPES buffer solution and the linear line used for the determination of the LoD.

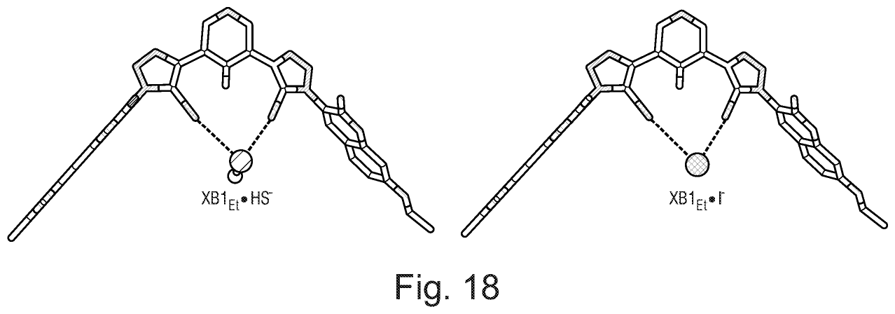

FIG. 18. DFT optimised structures of XB1_{Et} model receptor halogen bonded to HS⁻ (top) and I⁻ (bottom) anions. The XB interactions are sketched as purple dashed lines.

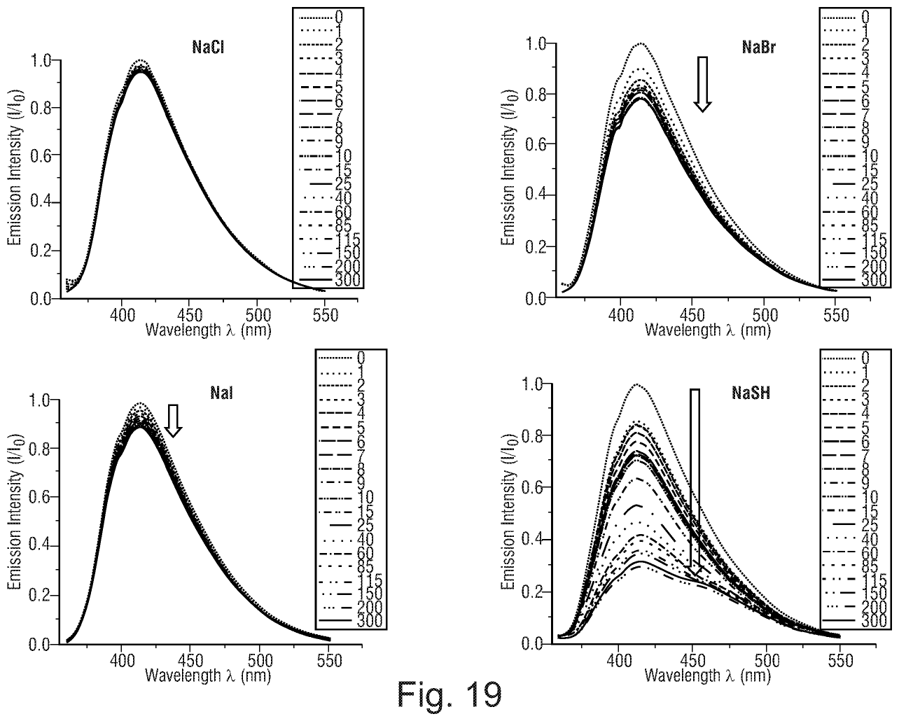

FIG. 19. Fluorescence response upon addition of NaCl, NaBr, NaI and NaSH to XB4 in unbuffered water at 298 K.

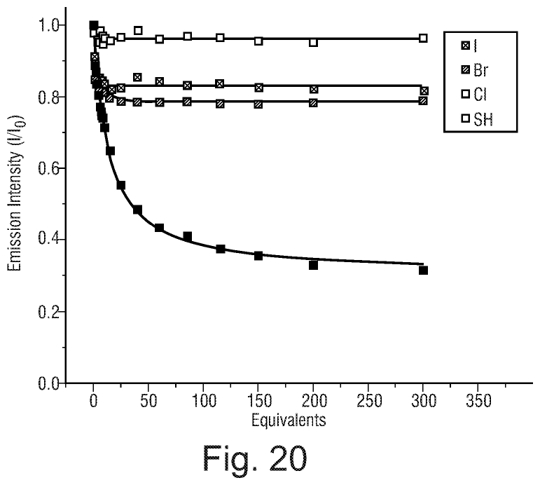

FIG. 20. The relative change in emission intensity at 412 nm upon addition of NaCl, NaBr, NaI and NaSH to XB4 in unbuffered water at 298 K.

### 1. MATERIALS AND METHODS

### Materials

The commercially available reagents and solvents used in these examples were used as received without further purification. All dry solvents were thoroughly degassed with N₂, dried through a Mbraun MPSP-800 column and used immediately. Water used was deionized and passed through a Milli-Q® Millipore machine for microfiltration. TBTA (tris(benzyltriazolemethyl)amine) was prepared according to reported procedures.

### Methods

Chromatography was undertaken using silica gel (particle size: 40-63 μm) or preparative TLC plates (20×20 cm, 1 cm silica thickness).

NMR spectra were recorded on Bruker AVIII HD Nanobay 400 MHZ, Bruker AVIII 500 MHz and Bruker AVIII 500 MHz spectrometers.

Low resolution electrospray 62haracteri mass spectrometry (ESI-MS) was performed using the Waters Micromass LCT for 62haracterization of compounds previously reported in the literature, and high resolution ESI-MS was recorded using Bruker microTOF spectrometer for novel compounds.

Fluorescence titration experiments were performed using a Horiba Duetta at 293 K. The host molecules (XB1-3, and HB1-3) were dissolved in 10 mM HEPES aqueous buffer solution at pH 7.4 at a concentration of 10 μM. Na salts of the appropriate anion were dissolved in the solution of the host molecule to obtain a concentration. Aliquots of the anion solution were added to 1.00 mL of the host solution in a quartz cuvette, where the sample was then thoroughly mixed before fluorescence spectra were recorded. Association constants were obtained by global analysis using BindFit, using a host-guest 1:1 binding model.

The anion sensing properties were investigated in pH 7.4 aqueous HEPES buffer solution using fluorescence spectroscopy. At this pH value the protonated forms XB3H⁺ and HB3H⁺ are the dominant receptor species present in the aqueous solution. The amine pKₐ of XB3 (10.29) and HB3 (9.98) were experimentally determined by monitoring changes in fluorescence with pH. (FIG. 16).

Quantum calculations were carried out with Gaussian 16 (DFT calculations), while atomic RESP charges derivatisation was carried out with Gaussian 09. The classical force field calculations, including the Molecular Mechanics (MM) energy minimisations and Molecular Dynamics (MD) simulations, were carried out with AMBER 18.

DFT calculations: The HS⁻ and I⁻ associations of the XB1, XB2 and XB3H⁺ acyclic model receptors were optimised with the M06-2X functional. The iodine centres were described with the Def2-TZVPD basis sets, while the Def2-TZVP basis set was used for the remaining elements. Water solvation effects were accounted for with the Conductor-like Polarisable Continuum Model (CPCM). The free model receptors and anions were also optimised in the same conditions. The absence of imaginary frequencies on the vibrational frequency analyses confirmed that all stationary point geometries were local minima on the potential energy surface.

Analyses of the XB interactions on the anion associations were carried out with Natural Bond Orbitals using NBO6 and with Quantum Theory of Atoms in Molecules using MultiWFN 3.6. MultiWFN was also used to calculate the distribution of electrostatic potential on the electron density surface (Vs) of the model receptors.

The Molecular modelling studies in solution were carried out with complete XB1, XB2 and XB3H⁺ associated with HS⁻ and I⁻ anions using the General AMBER Force Field (GAFF) force field parameters to describe the acyclic receptors and the HS⁻ anion. The molecular mechanics calculations were preceded by the derivatisation of Restrained Electrostatic Potential (RESP) charges as follows: the XB1, XB2 and XB3H⁺ receptors were optimised at the B3LYP/6-31G (d) level of theory, followed by a single point calculation at HF/6-31G (d) in which the RESP atomic charges were obtained using the Gaussian lops (6/33=2, 6/41=4, 6/42=6), in agreement with GAFF's development. In both the optimisation and single point, the iodine centres were described with the aug-cc-pVDZ-PP basis set. The derivatisation of the HS⁻ RESP charges was carried out following the same method. The iodide was described with a net charge of −1 and suitable vdW parameters.

### Synthesis of Coumarin-Containing Compounds

The synthesis of XB1-3 and HB1-3 is outlined in Scheme 1 below.

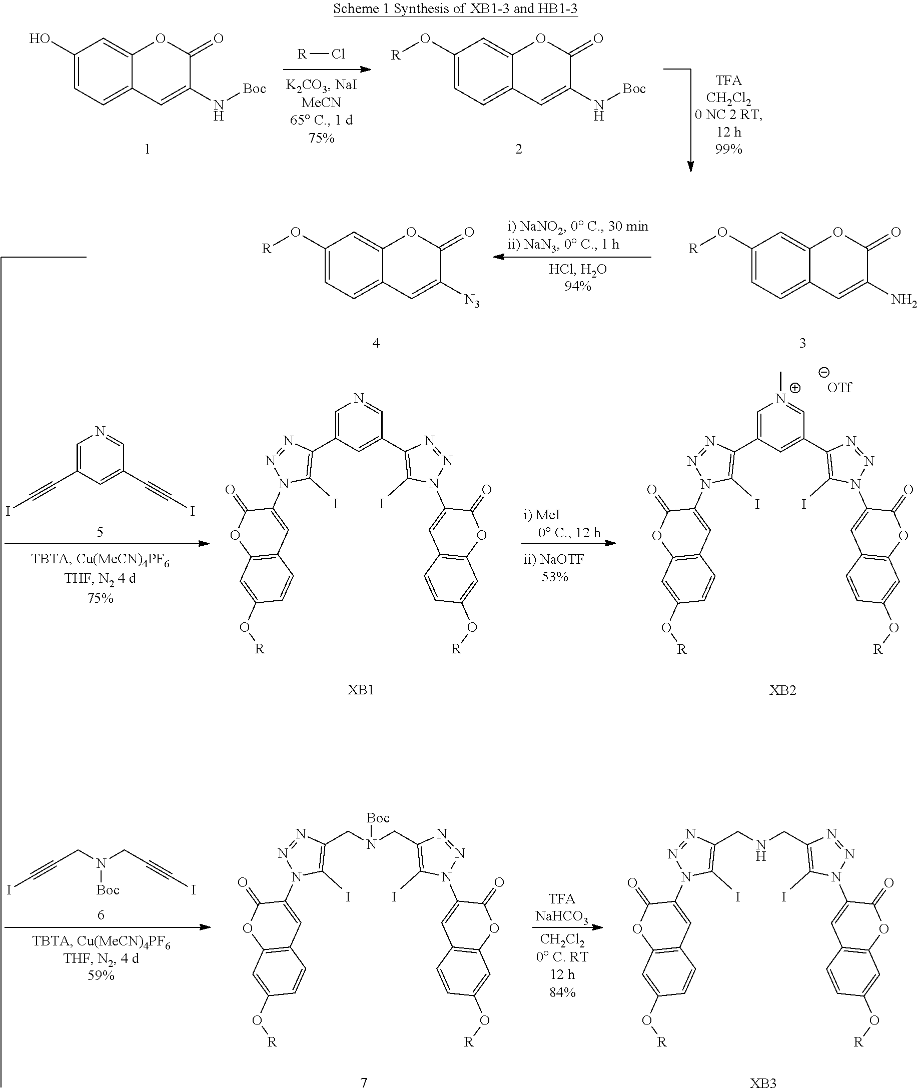

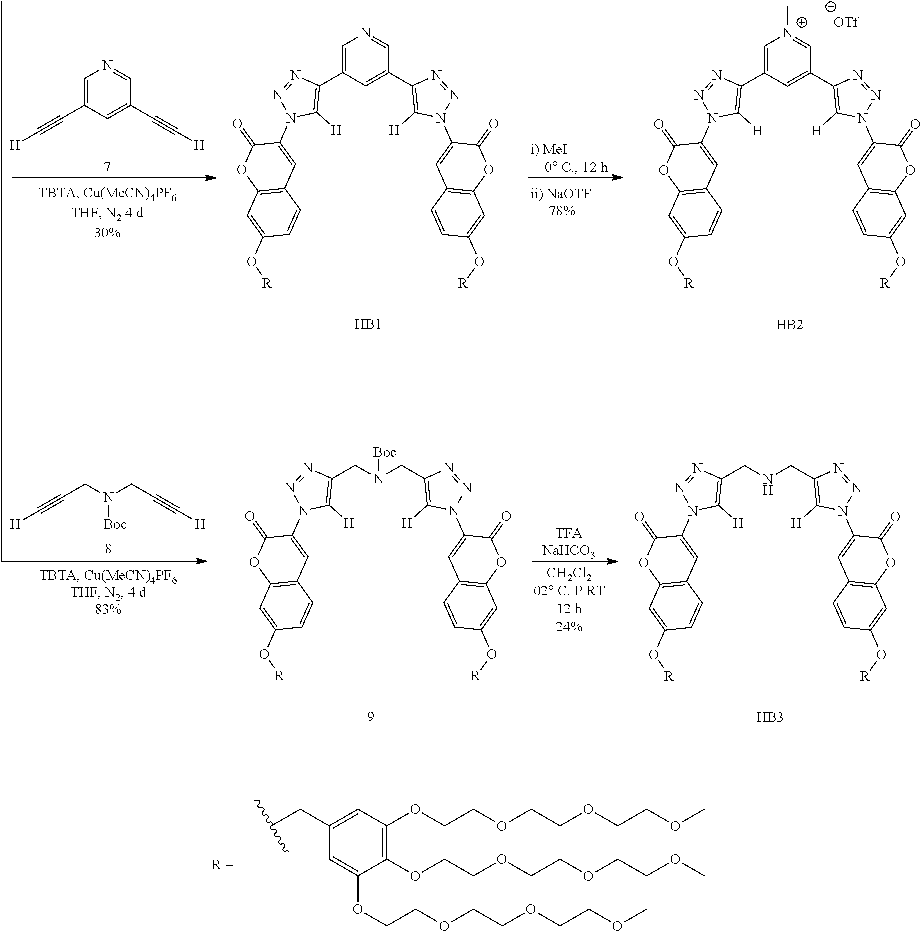

The synthetic steps are as follows: 7-hydroxycoumarin 1 was reacted with 1.2 equivalents of the appropriate chloro-alkyl/benzyl PEG functionalized derivative under basic conditions and in the presence of NaI to afford 2 in 75% yield. Boc deprotection of 2 with TFA produced amine 3 which upon sequential diazotization using NaNO₂ and addition of NaN₃ in acidic aqueous solution afforded coumarin-azide 4 in yields of 94%. The copper (I) catalysed azide-iodoalkyne cycloaddition (CuAAC) reaction of two equivalents of coumarin azide 4 with one equivalent of 3,5-bis-iodoalkyne pyridine 5 or N-Boc-protected bis-iodoalkyne 6 in the presence of a [Cu(CH₃CN)₄]PF₆ catalyst and TBTA afforded XB1 and Boc-protected bis-triazole derivative 7 in 75% and 59% yields, respectively. Methylation of XB1 to produce the pyridinium receptor XB2 was achieved via reaction with excess methyl iodide, followed by anion exchange with sodium trifluoromethanesulfonate. Boc deprotection of 7 was achieved using TFA to afford XB3.

Analogous synthetic methods were used to prepare the corresponding hydrogen bonding hosts HB1-3 (Scheme 1). All receptors were characterized by 1H and 13C NMR spectroscopy, high resolution mass spectrometry, UV-Vis and fluorescence spectroscopy:

### XB1

1H NMR (500 MHZ, CDCl₃) δ 9.39 (d, J=2.1 Hz, 2H, Hₕ), 9.02 (d, J=2.1 Hz, 1H, H_{g}), 8.10 (s, 2H, H_{f}), 7.60 (d, J=8.7 Hz, 2H, Hₑ), 7.08 (dd, J=8.7, 2.4 Hz, 2H, H_{d}), 7.03 (d, J=2.4 Hz, 2H, H_{c}), 6.71 (s, 4H, Hₐ), 5.11 (s, 4H, H_{b}), 4.22-4.17 (m, 12H, OCH₂), 3.90-3.87 (m, 8H, OCH₂), 3.83-3.81 (m, 4H, OCH₂), 3.77-3.74 (m, 12H, OCH₂), 3.71-3.66 (m, 24H, OCH₂), 3.58-3.56 (m, 12H, OCH₂), 3.40-3.39 (m, 18H, OCH₃); ¹³C NMR (126 MHz, CDCl₃) δ 163.75, 156.38, 156.24, 153.01, 148.36, 147.45, 142.79, 138.63, 132.87, 130.59, 130.49, 125.90, 120.63, 114.70, 111.31, 107.26, 102.17, 72.36, 71.95, 71.93, 70.94, 70.82, 70.69, 70.56, 70.54, 70.52, 69.71, 68.96, 59.05; Fluorescence (H₂O, 10 mM HEPES): λₑₓ=344 nm; λₑₘ=414 nm; HRMS (ESI+ve) m/z=1938.53697 ([M+H]⁺, C₈₃H₁₁₀N₇O₃₀I₂, calc=1938.53810)

### HB1

¹H NMR (500 MHZ, CDCl₃) δ 9.17 (d, J=2.1 Hz, 2H, Hᵢ), 9.06 (s, 2H, H_{g}), 8.76 (t, J=2.2 Hz, 1H, Hₕ), 8.64 (s, 2H, H_{f}), 7.63 (d, J=8.8 Hz, 2H, Hₑ), 7.06 (dd, J=8.8, 2.4 Hz, 2H, Hₐ), 6.99 (d, J=2.4 Hz, 2H, H_{c}), 6.68 (s, 4H, Hₐ), 5.07 (s, 2H, H_{b}), 4.19-4.16 (m, 12H, OCH₂), 3.89-3.85 (m, 8H, OCH₂), 3.81-3.79 (m, 4H, OCH₂), 3.76-3.70 (m, 12H, OCH₂), 3.70-3.62 (m, 24H, OCH₂), 3.56-3.54 (m, 12H, OCH₂), 3.38-3.37 (m, 18H, OCH₃); ¹³C NMR (126 MHZ, CDCl₃) δ 163.00, 156.27, 154.76, 152.95, 146.44, 144.49, 138.40, 134.27, 130.95, 130.41, 130.29, 126.72, 121.53, 120.16, 114.87, 111.80, 107.18, 101.89, 72.38, 71.94, 71.92, 70.89, 70.80, 70.65, 70.57, 70.51, 70.49, 69.73, 68.89, 59.02, 50.56; Fluorescence (H₂O, 10 mM HEPES): λₑₓ=342 nm; λₑₘ=498 nm; HRMS (High resolution ESI+ve) m/z=1686.7401 ([M+H]⁺, C₈₃H₁₁₁N₇O₃₀, calc=1686.7448)

### XB2

¹H NMR (400 MHZ, CDCl₃) δ 9.59 (s, 3H, H_{h,g}), 8.11 (s, 2H, Hᵢ), 7.59 (d, J=8.8 Hz, 2H, Hₑ), 7.06 (dd, J=8.8, 2.3 Hz, 2H, H_{d}), 6.92 (d, J=2.3 Hz, 2H, H_{c}), 6.61 (s, 4H, Hₐ), 5.04 (s, 4H, H_{b}), 4.76 (s, 3H, Hᵢ), 4.13-4.04 (m, 12H, OCH₂), 3.83-3.78 (m, 8H, OCH₂), 3.78-3.65 (m, 32H, OCH₂), 3.64-3.59 (m, 8H, OCH₂), 3.59-3.54 (m, 4H, OCH₂), 3.54-3.48 (m, 8H), 3.33 (s, 6H, OCH₃), 3.30 (s, 12H, OCH₃); ¹³C NMR (126 MHZ, CDCl₃) δ 164.02, 156.70, 156.25, 152.41, 143.49, 142.97, 142.64, 137.68, 133.17, 130.95, 130.35, 129.30, 128.22, 121.75, 119.87, 119.20, 114.80, 111.19, 106.57, 102.14, 86.11, 72.33, 71.97, 71.92, 70.93, 70.84, 70.71, 70.69, 70.49, 69.75, 68.57, 58.96, 58.92, 53.80, 49.40; ¹⁹F NMR (377 MHZ, CDCl₃) δ −77.86; Fluorescence (H₂O, 10 mM HEPES): λₑₓ=344 nm; λₑₘ=408 nm; HRMS (ESI+ve) m/z=1952.5531 ([M]⁺, C₈₄H₁₁₂I₂N₇O₃₀+, calc=1952.5537)

### HB2

¹H NMR (500 MHZ, CDCl₃) δ 9.43 (s, 2H, Hₕ), 9.26 (s, 2H, H_{g}), 9.25 (s, 1H, Hₕ), 8.44 (s, 2H, H_{f}), 7.59 (d, J=8.8 Hz, 2H, Hₑ), 7.09 (dd, J=8.8, 2.4 Hz, 2H, Hₐ), 6.85 (d, J=2.4 Hz, 2H, H_{c}), 6.55 (s, 4H, Hₐ), 4.91 (s, 4H, H_{b}), 4.68 (s, 3H, Hⱼ), 4.17-4.13 (m, 8H, OCH₂), 4.01-3.98 (m, 4H, OCH₂), 3.83-3.80 (m, 8H, OCH₂), 3.77-3.70 (m, 22H, OCH₂), 3.69-3.67 (m, 7H, OCH₂), 3.65-3.61 (m, 8H, OCH₂), 3.60-3.56 (m, 3H, OCH₂), 3.52-3.48 (m, 8H, OCH₂), 3.29 (s, 11H, OCH₃), 3.28 (s, 6H, OCH₃); ¹³C NMR (126 MHZ, CDCl₃) δ 162.86, 155.95, 154.75, 152.35, 141.35, 141.05, 137.67, 135.48, 134.74, 130.81, 130.62, 130.57, 124.32, 122.14, 119.91, 119.59, 114.70, 111.49, 105.85, 101.43, 72.14, 71.95, 71.90, 70.91, 70.76, 70.71, 70.68, 70.58, 70.53, 70.50, 70.34, 69.73, 68.43, 58.93, 58.81, 48.72; Fluorescence (H₂O, 10 mM HEPES): λₑₓ=344 nm; λₑₘ=424 nm; HRMS (ESI+ve) m/z=1700.7563 ([M]⁺, C₈₄H₁₁₄N₇O₃₀+, calc=1700.7605)

### XB3

¹H NMR (500 MHZ, CDCl₃) δ 7.98 (s, 2H, H_{f}), 7.54 (d, J=8.7 Hz, 2H, Hₑ), 7.03 (dd, J=8.7, 2.4 Hz, 2H, Hₐ), 6.98 (d, J=2.4 Hz, 2H, H_{c}), 6.67 (s, 4H, Hₐ), 5.07 (s, 4H, H_{b}), 4.20-4.14 (m, 12H, OCH₂), 4.04 (s, 4H, H_{g}), 3.86-3.84 (m, 8H, OCH₂), 3.81-3.78 (m, 4H, OCH₂), 3.74-3.71 (m, 12H, OCH₂), 3.68-3.62 (m, 24H, OCH₂), 3.56-3.52 (m, 12H, OCH₂), 3.37 (s, 6H, OCH₃), 3.37 (s, 12H, OCH₃); ¹³C NMR (126 MHZ, CDCl₃) δ 163.56, 156.36, 156.11, 153.01, 150.35, 142.31, 138.64, 130.62, 130.38, 120.77, 114.55, 111.37, 107.28, 102.09, 82.24, 72.37, 71.96, 71.94, 70.92, 70.83, 70.70, 70.57, 70.55, 70.53, 69.72, 68.97, 59.05, 44.10, 29.74; Fluorescence (H₂O, 10 mM HEPES): λₑₓ=342 nm; λₑₘ=412 nm; HRMS (ESI+ve) m/z=1904.55375 ([M+H]⁺, C₈₀H₁₁₂I₂N₇O₃₀, calc=1904.54083)

### HB3

¹H NMR (500 MHZ, CDCl₃) δ 8.58 (d, 2H, H_{g}), 8.51 (d, 2H, Hᵢ), 7.57 (d, J=8.7 Hz, 2H, Hₑ), 7.03 (dd, J=8.7, 2.4 Hz, 2H, Hₐ), 6.96 (d, J=2.4 Hz, 2H, H_{c}), 6.67 (s, 4H, Hₐ), 5.05 (s, 4H, H_{b}), 4.19-4.11 (m, 12H, OCH₂), 3.87-3.82 (m, 8H, OCH₂), 3.81-3.77 (m, 4H, OCH₂), 3.74-3.70 (m, 12H, OCH₂), 3.67-3.62 (m, 24H, OCH₂), 3.55-3.52 (m, 12H, OCH₂), 3.49 (s, 4H, Hₕ), 3.37-3.36 (m, 18H, OCH₃); ¹³C NMR (126 MHZ, CDCl₃) δ 162.67, 156.13, 154.60, 152.99, 152.72, 145.88, 138.59, 137.83, 136.65, 133.79, 130.77, 130.00, 123.22, 120.51, 114.59, 111.83, 107.27, 106.68, 101.78, 72.36, 72.27, 71.96, 71.94, 70.83, 70.78, 70.72, 70.70, 70.57, 70.55, 70.52, 69.83, 69.72, 68.96, 68.86, 65.33, 59.05, 43.66, 31.94, 29.71, 22.71, 14.14, 1.03; Fluorescence (H₂O, 10 mM HEPES): λₑₓ=342 nm; λₑₘ=484 nm; HRMS (High resolution ESI+ve) m/z=1652.76046 ([M+H]⁺, C₈₀H₁₁₄N₇O₃₀, calc=1652.76113)

### Synthesis of XB4

The synthesis of XB4 is outlined in Scheme 2 below:

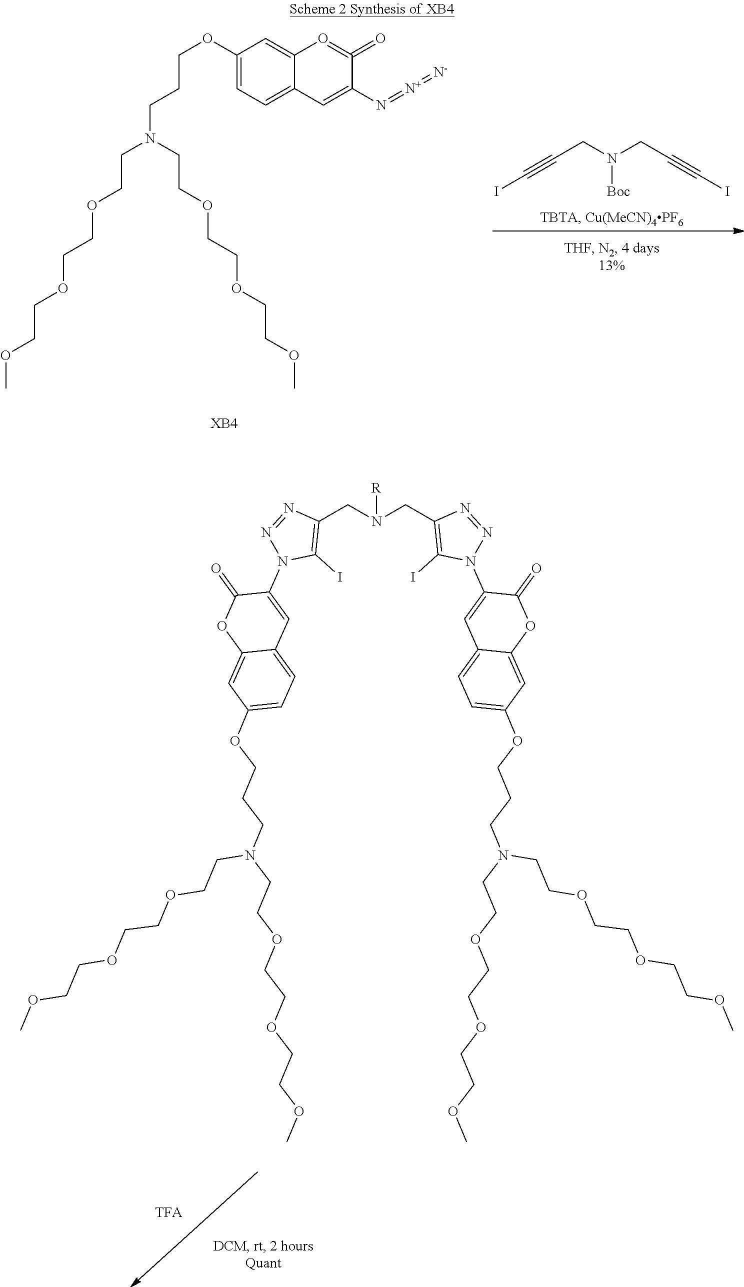

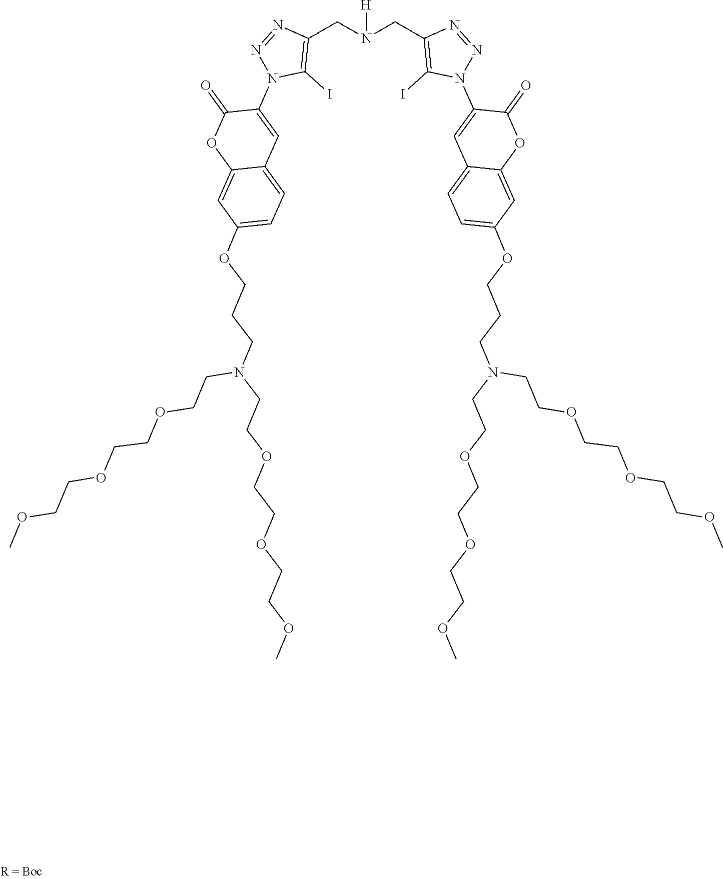

Just over two equivalents (2.2) of the coumarin azide was added to one equivalent of the bis-alkyne with catalytic quantities of Cu^{I} and TBTA. The reaction was left to stir for 3 days at room temperature under N₂, and purification was achieved by preparative TLC using 5% MeOH in DCM to give the desired receptor in a 13% yield. Quantitative deprotection of the Boc-group using TFA afforded receptor XB4.

### Synthesis of BODIPY-Containing Compounds

The synthesis of XB-BDP-PEG and HB-BDP-PEG is outlined in Scheme 3 below:

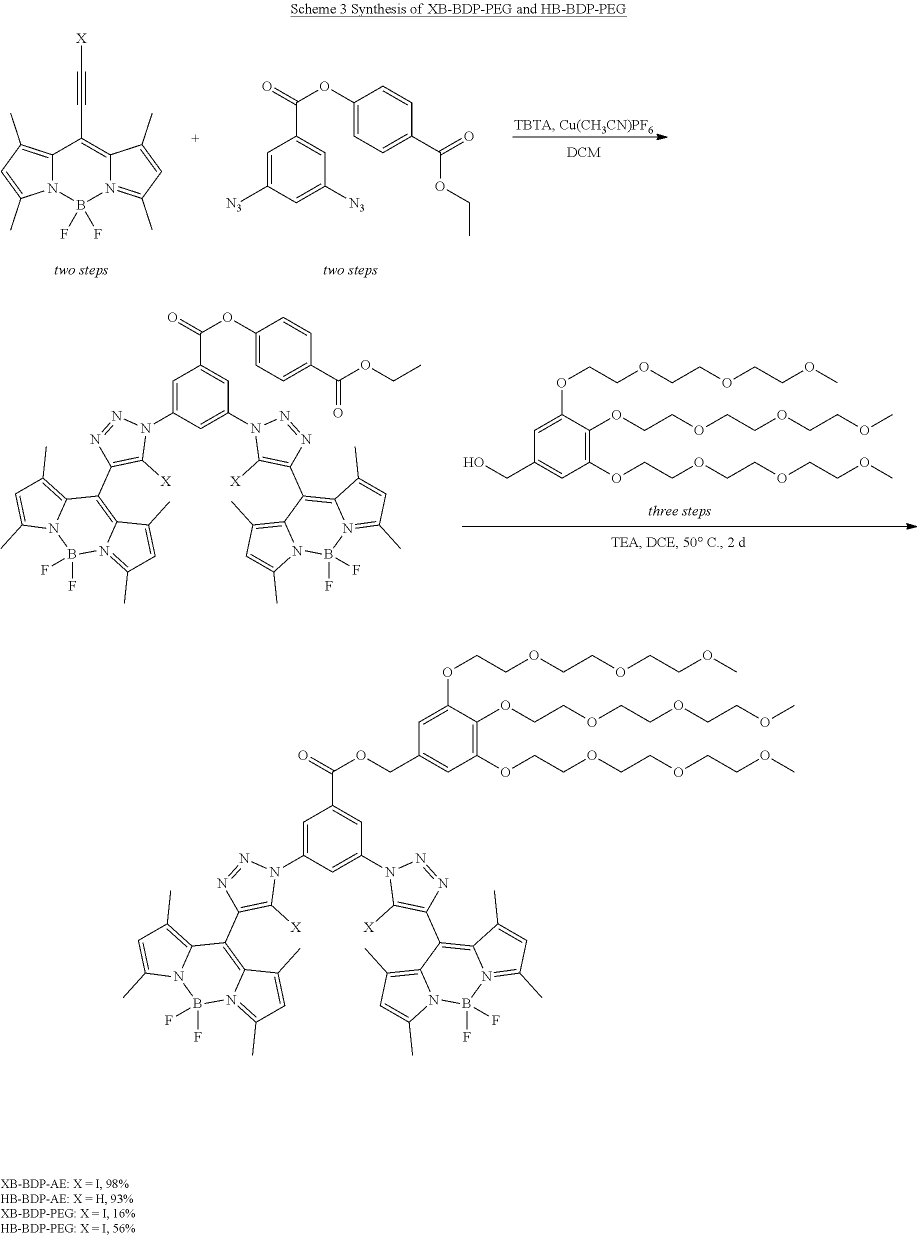

The synthesis of the BODIPY-containing compounds is largely analogous to the synthetic steps for the coumarin-containing compounds. The modular synthesis afforded XB-BDP-AE and HB-BDP-AE (for surface-immobilisation; obtained in 5 overall synthetic steps each in good yields) as well as water-soluble receptors XB-BDP-PEG and HB-BDP-PEG (for comparative solution-phase studies; obtained in three further synthetic steps in moderate yields).

### 2. RESULTS AND DISCUSSION

### Fluorescence Studies

The fluorescence emission, following excitation at 342 nm, was monitored upon the addition of sodium hydrosulfide and halide salts. Remarkably, the fluorescence intensity of XB1 was significantly quenched by 60% in the presence of 10 equivalents of HS⁻, while I⁻, Br⁻ and Cl⁻ induced no intensity diminutions (FIGS. 1, 2 and 9). In stark contrast, HB1 exhibited only minor fluorescence perturbations (FIG. 10) suggesting very weak binding which notably highlights the superior capability of XB donor motifs for anion recognition and sensing in water in comparison to HB donor analogues.

Similarly, the addition of HS⁻ XB2 and XB3H⁺ caused notable quenching, 45% and 38% respectively, whereas no change was observed with Cl⁻. With XB2, both Br⁻ and I⁻ were sensed (FIG. 11), with the heavier halide also being detected by XB3H⁺ (FIG. 13). In contrast, the hydrogen bonding analogue HB3H⁺ was found to have no fluorescent response to any anion (FIG. 14). However, HB2 exhibited a selective fluorescence intensity decrease of 22% with addition of 100 equivalents of I⁻ (FIG. 12).

XB-BDP-PEG and HB-BDP-PEG are generally water soluble. Any observed fluorescence quenching in water could be readily circumvented by addition of a small amount of organic co-solvent. Preliminary solution-phase fluorescence sensing studies of both receptors were thus carried out in 70% water/30% acetonitrile. As shown in FIGS. 7 and 8, XB-BDP-PEG responds to the HS⁻ anion via strong emission quenching of up to ≈65%. Smaller, but notable, perturbations were also observed for I⁻ and SCN⁻, however these anions are unlikely to co-exist with HS⁻ in most analytical samples. Importantly, both CI and Br induced only very small responses.

Saliently, the response of the analogous HB-BDP-PEG probe, which exhibits HB, was significantly diminished for all anions, in particular HS⁻, highlighting the superiority of the XB interaction for HS⁻ recognition and sensing.

Fluorescence titrations of XB4 were carried out with chloride, bromide, iodide and hydrosulfide. All anions produced a “switching off” of coumarin fluorescence as more equivalents of anion were added, with the exception of chloride, as shown in FIG. 19.

### Bindfit Analysis

Bindfit analysis^{23,24} of the titration data for XB and HB receptors was used to determine 1:1 host: guest stoichiometric anion association constants, as shown below in Table 1.

TABLE 1
Anion association constants and limits of detection
of HS⁻ for XB1-3H⁺ and HB1-3H⁺

| | Association constant Kₐ [M⁻¹]^{[a][b]} | | | | LoD |
|---|---|---|---|---|---|
| Compound | Cl⁻ | Br⁻ | I⁻ | HS⁻ | [μM] |
| XB1 | NB | NB | NB | 16500 (1600) | 14.3 |
| XB2 | NB | 60800 | 103000 (24000) | 158000 (31000) | 6.7 |
| | | (7800) | | | |
| XB3H⁺ | NB | NB | 46000 (14000) | 5600 (530) | 24.6 |
| HB1 | NB | NB | NB | NB | NB |
| HB2 | NB | NB | 6080 (550) | NB | NB |
| HB3H⁺ | NB | NB | NB | NB | NB |

NB—No binding observed
LoD—Limit of Detection for HS−
^{[a]}Anion association constants (with errors in brackets), determined using Bindfit analysis of the fluorescence titration data monitoring averaged λₘₐₓ (400-420 nm)
^{[b]}10 μM receptors in 10 mM aqueous HEPES buffer solution, pH = 7.4.

Notably, neutral XB1 behaves as an exclusive chemosensor for HS⁻, exhibiting strong and selective recognition for HS⁻ over the halides, which are not bound. As expected, the magnitude of association to HS⁻ by positively charged pyridinium receptor XB2 is significantly greater compared to neutral XB1 due to favourable electrostatic interactions. However, the increase in binding strength comes at the expense of a diminished degree of selectivity for HS⁻ as XB2 also binds Br⁻ and in particular I⁻ strongly. XB3H⁺ exhibits impressive HS⁻ selectivity over the lighter halides but forms the strongest association with I⁻. To rule out the possibility of HS⁻ reacting with XB1, electrospray mass spectrometry (EMS) experiments monitoring 10 μM XB1 in 10 mM HEPES aqueous buffer (7.4 pH) in the presence of excess equivalents of HS⁻ were conducted, no evidence of reaction was observed (FIGS. S6-1). Furthermore, titrations of buffered 10 μM receptor XB3 against NaOH revealed no change in fluorescence (FIGS. S3-5).

Importantly, the limits of detection (LoD) for HS⁻ for XB1-3 were calculated to be in the low micromolar range (Table 1, FIG. 17), comparable to chemodosimeter probes used for detecting H₂S in aqueous environments and in vitro studies.²⁵

BindFit analysis for each anion to determine binding isotherms (FIG. 20), and 1:1 stoichiometric association constants, shown in Table 2, was also conducted for XB4.

TABLE 2
Anion association constants of XB4 in unbuffered water at 298 K.

| Anion | Association constant Kₐ[M⁻¹]^{[a]} |
|---|---|
| Cl⁻ | N.B. |
| Br⁻ | >10⁵ |
| I⁻ | >10⁵ |
| HS⁻ | 7500 (660) |

^{[a]}Errors are in parentheses and are all <10%
N.B. No Binding

Importantly, Table 2 shows XB4 exhibits very strong binding for the heavier halides Br⁻ and I⁻ but not for Cl⁻. The anion association constants follow a Hofmeister bias selectivity trend, I⁻˜Br⁻>>Cl⁻, the binding affinity is greatest with the least hydrated species. The enthalpy of hydration for chloride is reported as ΔH⁰_{hyd}=−390 KJ mol⁻¹ and ΔH⁰_{hyd}=−330 KJ mol⁻¹ for hydrosulfide, meaning desolvation and subsequent binding of the hydrosulfide anion within the hydrophobic XB binding site is more favourable, as reflected in the association constant values.

### Reversibility Studies

In comparison to chemodosimeters, XB1-3 are capable of acting as reversible chemosensors. Reversibility studies were carried out by removal of HS⁻ using Zn(OTf)₂. Upon addition of the zinc salt to fluorescence HS⁻ quenched solutions of XB1-3, the respective receptor's fluorescence returned to near non-quenched emissions as ZnS precipitated. Importantly, this proves the receptors' chemosensing reversible capability (FIG. 15).

Density functional theory (DFT) calculations

The XB interactions between HS⁻ and I⁻ with XB1, XB2 and XB3H⁺ were evaluated using DFT calculations, at the M06-2X/Def2-TZVP theory level, with the iodine centres being described with the Def2-TZVPD basis set. Water solvation effects were taken into account using the CPCM solvent model. Due to the sizes of the receptors, their TEG substituted aryl moieties were replaced with ethyl groups, affording XB1_{Et}, XB2_{Et} and XB3_{Et}H⁺. These optimised structures are presented in FIG. 3 for the HS⁻ and I⁻ associations with XB2_{Et} and XB3_{Et}H⁺, while FIG. 18 shows the binding scenarios for the anion associations with XB1_{Et}.

16

### Natural Bond Orbital (NBO) Analysis

The strength of the XB interactions between the model receptors and HS⁻ and I⁻ was assessed with Natural Bond Orbital (NBO) analysis,²⁶ including Second Order perturbation theory interaction energies (E²), and variations in orbital occupancy. The E² values for the interactions between the C—I antibonding orbitals of model receptors and the anions' lone pairs orbitals (n_{A}→σ*_{C-I}) are summarised in Table 3:

TABLE 3
Natural Bond Orbital analysis of the anion
associations of XB1_{Et,} XB2_{Et} or XB3_{Et}H⁺.

| | | | | Change in electron | |
|---|---|---|---|---|---|
| | | E² (kcal | | occupancy (e⁻)^{c} | |
| Receptor | Anion | mol⁻¹)^{a} | WBI^{b} | σ*_{C—I} | n_{A} |
| XB1_{Et} | HS⁻ | 17.45; 17.44 | 0.12; 0.12 | 0.16 | −0.20 |
| | I⁻ | 9.19; 9.19 | 0.07; 0.07 | 0.09 | −0.11 |
| XB2_{Et} | HS⁻ | 21.16; 21.16 | 0.14; 0.14 | 0.20 | −0.24 |
| | I⁻ | 10.47; 10.48 | 0.08; 0.08 | 0.10 | −0.13 |
| XB3_{Et}H⁺ | HS⁻ | 22.08; 22.00 | 0.15; 0.15 | 0.21 | −0.25 |
| | I⁻ | 10.26; 10.34 | 0.08; 0.08 | 0.10 | −0.13 |

^{a}XB interactions' Second Order perturbation theory energies between the C—I antibonding orbitals of model receptors and the anions' lone pairs orbitals (n_{A}→σ*_{C—I});
^{b}Wiberg Bond Indices of the XB interactions;
^{c}Change in the electron occupancy in n_{A} and σ*_{C—I} orbitals upon anion binding of HS⁻ or I⁻.

In agreement with the shorter intermolecular distances computed for C—I ··· SH⁻ halogen bonds, the E² values indicate that the XB interactions are stronger in HS⁻ associations with the three model receptors, and naturally higher for the charged receptors XB2_{Et} and XB3_{Et}H⁺. Indeed, the E² values for the individual interactions with I⁻ range from 9.19 to 10.48 kcal mol⁻¹, significantly less than the values for HS⁻ between 17.44 and 22.08 kcal mol⁻¹. These stronger interactions also lead to a change in the σ*_{C-I} orbital occupancies upon anion binding, being more pronounced in the associations with HS⁻.

21

### Quantum Theory of Atoms in Molecules (QTAIM) Analysis

The XB interactions were also evaluated within the scope of the QTAIM,²⁷ with the analysis of their Bond Critical Points (BCP). Both the Potential energy density (V(r)) and Lagrangian kinetic energy density (G(r)) of BCP were used to estimate the XB interaction energies (see Table 4).²⁸

TABLE 4
Quantum Theory of Atoms in Molecules analysis of the XB interactions
in the anion associations of XB1_{Et}, XB2_{Et} or XB3_{Et}H⁺.

| | | ρ(r) | ∇²ρ(r) | V(r) | G(r) | E_{XB-V(r)} | E_{XB-G(r)} | |
|---|---|---|---|---|---|---|---|---|
| | | (×10, | (×10, | (×10, | (×10, | (kcal | (kcal | \|V(r)\|/G(r) |
| Receptor | Anion | ea₀⁻³)^{a} | ea₀⁻³)^{b} | Hartree)^{c} | Hartree)^{d} | mol⁻¹)^{e,g} | mol⁻¹)^{f,g} | ratio |
| XB1_{Et} | HS⁻ | 0.21; | 0.46; | −0.12; | 0.12; | −5.14; | −4.96; | 1.02; |
| | | 0.21 | 0.46 | −0.12 | 0.12 | −5.14 | −4.96 | 1.02 |
| | I⁻ | 0.13; | 0.32; | −0.07; | 0.07; | −2.92; | −3.11; | 0.92; |
| | | 0.13 | 0.32 | −0.07 | 0.07 | −2.92 | −3.11 | 0.92 |
| XB2_{Et} | HS⁻ | 0.24; | 0.49; | −0.14; | 0.13; | −5.94 | −5.50; | 1.06; |
| | | 0.24 | 0.49 | −0.14 | 0.13 | −5.94 | −5.50 | 1.06 |
| | I⁻ | 0.14; | 0.34; | −0.07; | 0.08; | −3.17; | −3.33; | 0.94; |
| | | 0.14 | 0.34 | −0.07 | 0.08 | −3.17 | −3.33 | 0.94 |
| XB3_{Et}H⁺ | HS⁻ | 0.25; | 0.50; | −0.15; | 0.13; | −6.19; | −5.65; | 1.08; |
| | | 0.25 | 0.49 | −0.14 | 0.13 | −6.13 | −5.61 | 1.08 |
| | I⁻ | 0.14; | 0.33; | −0.07; | 0.08; | −3.15; | −3.31; | 0.94; |
| | | 0.14 | 0.33 | −0.07 | 0.08 | −3.16 | −3.32 | 0.94 |

^{a}Electron density;
^{b}Laplacian of the density;
^{c}Potential energy density;
^{d}Lagrangian kinetic energy density;
^{e}Energies of the halogen bonds determined from V(r) as E_{XB-V(r)} = V(r) × 0.68;
^{f}Energies of the halogen bonds determined from G(r) as E_{XB-G(r)} = −G(r) × 0.67;
^{g}Both correlations were specifically developed to estimate the XB interaction energies involving iodine atoms.

The values of these two binding descriptors for a given model receptor with HS⁻ are ca. two times larger than with I⁻. Moreover, the energy values of both binding descriptors estimated for the six-anion associations correlate well with the corresponding E² ones (R²>=0.99), showing the equivalency between QTAIM and NBO analyses.

Moreover, the ratio between these two quantum descriptors, |V(r)|/G(r), permits to evaluate the nature of XB interactions, with values below 1 indicating a pure electrostatic interaction, values greater than 2 being typical of covalent bonds and the values in-between indicating an intermolecular interaction with an intermediate character between ionic and covalent bonds. The |V(r)|/G(r) ratios estimated for the XB interactions of XB1_{Et}, XB2_{Et} and XB3_{Et}H⁺ with I⁻ (see Table 4) are ca. 0.9, suggesting that they are highly electrostatic, while the interactions with HS⁻, with ratios around 1.1 indicate a slight increase of the covalency on all three receptor associations with this more nucleophilic anion. Equivalent conclusions can be drawn from the Wiberg Bond Indices, which range from 0.07 to 0.08 for I⁻ associations and from 0.12 to 0.14 for HS⁻ associations (Table 3). The differences found in the nature of the XB interactions between the model receptors and HS⁻ or I⁻ are also illustrated in FIG. 4, where the changes in electron density between XB3_{Et}H⁺ associations with HS⁻ or I⁻ and their individual components are plotted. The loss of electron density around the guest anion (blue orbital regions) is complemented with a gain of electron density along the iodotriazole binding units (pink areas).

22

### Intermolecular Binding Free Energies

The strength of the XB interactions was also evaluated by means of intermolecular binding free energies between XB1_{Et}, XB2_{Et} or XB3_{Et}H⁺ and I⁻ or HS⁻ estimated as the energy differences between the associations and the free model receptors and anions. The binding energies (ΔG^{SS}) gathered in Table 5, ranging between −8.79 and −4.19 kcal mol⁻¹ for the HS⁻ association and from −0.77 to −4.18 kcal mol⁻¹ for the I⁻ associations, corroborate the stronger binding affinity of these receptors for HS⁻, as suggested by the other quantum descriptors. Moreover, the predicted binding affinity between neutral receptor XB1_{Et} and I⁻ is almost negligible in agreement with the experimental inability of XB1 to bind this halide in water (see Table 1).

TABLE 5
Uncorrected electronic binding energies (Δε₀), Zero Point Corrections
(ΔZPE),^{a} thermal corrected binding energies (ΔE),^{b} binding enthalpies
(ΔH),^{c} binding entropies term contribution (TΔS),^{d} binding free energies
(ΔG_{bind}),^{e} and standard state binding free energies (ΔG^{SS})^{f} (in kcal mol⁻¹)
estimated from the DFT optimised structures of the HS⁻ and I⁻ associations with
XB1_{Et}, XB2_{Et} or XB3_{Et}H⁺, using the CPCM water solvent model.

| Receptor | Anion | Δε₀ | ΔZPE | ΔE | ΔH | TΔS | ΔG_{bind} | ΔG^{SS} |
|---|---|---|---|---|---|---|---|---|
| XB1_{Et} | HS⁻ | −14.88 | 1.15 | −13.31 | −13.91 | −11.60 | −2.30 | −4.19 |
| | I⁻ | −8.78 | 0.22 | −7.97 | −8.56 | −9.68 | 1.12 | −0.77 |
| XB2_{Et} | HS⁻ | −17.24 | 1.02 | −15.70 | −16.30 | −9.40 | −6.90 | −8.79 |
| | I⁻ | −10.22 | 0.17 | −9.40 | −10.00 | −7.71 | −2.29 | −4.18 |
| XB3_{Et}H⁺ | HS⁻ | −16.35 | 0.96 | −14.90 | −15.49 | −9.93 | −5.56 | −7.45 |
| | I⁻ | −9.20 | 0.22 | −8.40 | −8.99 | −8.46 | −0.53 | −2.42 |

^{a}ΔZPE is included in the ΔE, ΔH and ΔG terms;
^{b}ΔE = Δε₀ + ΔE_{Tot}, where ΔE_{Tot} accounts for the differences in the internal energy due to translational, rotational, vibrational and electronic motions;
^{c}ΔH = ΔE + ΔnRT, where n is −1 for a 1:1 host-guest systems, R is the ideal gas constant and T is the temperature (298.15 K);
^{d}ΔS = Δ_{Stranslational} + ΔSᵣₒₜₐₜᵢₒₙₐₗ + ΔS_{vibrational};
^{e}ΔG_{bind} = ΔH − TΔS
^{f}ΔG^{SS} is the free energy (ΔG_{bind}) including a −1.89 kcal mol⁻¹ correction which corresponds to the conversion from the standard state at 1 atm (1 mol per 24.46 L at 298.15 K) to 1M (1 mol/L at 298.15 K).

### Molecular Dynamics (MD)

Having ascertained the binding with DFT calculations the XB-based associations between HS⁻ and I⁻ and the entire receptors were investigated further by MD simulations carried out in periodic boxes of TIP3P water molecules. The receptors and HS⁻ were described with GAFF parameters^{29,3023,24} and RESP³¹ charges, while for I⁻, with a net charge of −1, Lennard-Jones 12-6 potential parameters were used.³²

Regardless of the anion-receptor association, the XB interactions are maintained nearly throughout the whole simulation time with average dimensions listed in Table 6.

TABLE 6
Average dimensions, with standard deviations, of the XB
interactions assessed in the MD simulations of the associations of XB1,
XB2 or XB3H⁺ with HS⁻ and I⁻ in water.^{a}

| | | I . . . A (Å) | | C—I . . . A (°) | |
|---|---|---|---|---|---|
| Receptor | Anion | Avg ± SD | Range | Avg ± SD | Range |
| XB1 | HS⁻ | 3.262 ± 0.130 | [2.402, 4.448] | 174.2 ± 2.8 | [157.9, 180.0] |
| | I⁻ | 4.245 ± 0.283 | [3.193, 10.238] | 172.5 ± 4.7 | [94.6, 180.0] |
| XB2 | HS⁻ | 3.181 ± 0.107 | [2.401, 7.816] | 174.7 ± 2.6 | [122.7, 180.0] |
| | I⁻ | 4.166 ± 0.268 | [3.495, 11.844] | 172.6 ± 5.3 | [95.9, 180.0] |
| XB3H⁺ | HS⁻ | 3.249 ± 0.125 | [2.823, 4.224] | 174.2 ± 2.8 | [151.7, 180.0] |
| | I⁻ | 4.254 ± 0.310 | [3.551, 10.838] | 171.3 ± 5.9 | [106.4, 180.0] |

^{a}N = 400000 using the four independent MD runs of 50 ns (50000 frames) with the two XB interactions treated together.

As predicted by DFT calculations, the XB interactions are almost linear with C—I ··· SH⁻ and C—I ··· I⁻ average angles of ca. 174° and 172°, respectively. However, the I ··· SH⁻ and I ···I⁻ average distances are systematically longer than the ones calculated by DFT by ca. 0.5 Å. In addition, the halogen bonded HS⁻ and I⁻ anions are surrounded by solvation water molecules as shown for the associations with XB₃H⁺ in FIG. 5. Furthermore, the TEG chains exhibited a large conformational flexibility along the MD runs, which suggest that they have no role in the shielding of the guest anion from the solvating water molecules. In other words, the PEG chains contribute only for the water solubilisation of three bis-iodotriazole XB-based receptors. A small decrease on the average number of the anions' surrounding water molecules is observed when compared with the freely solvated anions, from 10.6 to 7.9 for HS⁻ and from 7.7 to 6.0 for I⁻ for an anion-water distance cut-off of 3.5 Å. This result suggests that the water molecules play an important role on the anion recognition.

While specific embodiments of the invention have been described herein for the purpose of reference and illustration, various modifications will be apparent to a person skilled in the art without departing from the scope of the invention as defined by the appended claims.

### REFERENCES

1. Evans, N. H. & Beer, P. D. Advances in anion supramolecular chemistry: From recognition to chemical applications. Angewandte Chemie—International Edition vol. 53 11716-11754 (2014).
2. Wang, R. Two's company, three's a crowd: can H 2 S be the third endogenous gaseous transmitter? (2002).
3. Wang, R. Physiological implications of hydrogen sulfide: A whiff exploration that blossomed. Physiological Reviews 92, 791-896 (2012).
4. Polhemus, D. J. & Lefer, D. J. Emergence of hydrogen sulfide as an endogenous gaseous signaling molecule in cardiovascular disease. Circulation Research vol. 114 730-737 (2014).
5. Wallace, J. L. & Wang, R. Hydrogen sulfide-based therapeutics: Exploiting a unique but ubiquitous gasotransmitter. Nature Reviews Drug Discovery vol. 14 329-345 (2015).
6. Singh, S. & Lin, H. Hydrogen Sulfide in Physiology and Diseases of the Digestive Tract. Microorganisms 3, 866-889 (2015).
7. D. Wu, W. S. M. W. S. L. A. J. and Y. L. Hydrogen sulfide in cancer: Friend or foe? Nitric Oxide—Biol. Chem. 50, 38-45 (2015).
8. H. Tien Ngo, X. L. and K. A. J. Anion recognition and sensing with Zn(II)-dipicolylamine complexes. Chem. Soc. Rev 41, 4928-4965 (2012).
9. Langton, M. J., Serpell, C. J. & Beer, P. D. 2016. Angewandte Chemie 128, 2012-2026 (2016).
10. Lawrence, N. S., Davis, J. & Compton, R. G. Analytical strategies for the detection of sulfide: a review. Talanta vol. 52 www.elsevier.com/locate/talanta (2000).
11. Lin, V. S. & Chang, C. J. Fluorescent probes for sensing and imaging biological hydrogen sulfide. Current Opinion in Chemical Biology vol. 16 595-601 (2012).
12. Hartle, M. D. et al. A Synthetic Supramolecular Receptor for the Hydrosulfide Anion. Angewandte Chemie 128, 11652-11656 (2016).
13. Lau, N., Zakharov, L. N. & Pluth, M. D. Modular tripodal receptors for the hydrosulfide (HS−) anion. Chemical Communications 54, 2337-2340 (2018).
14. Vázquez, J. & Sindelar, V. Supramolecular binding and release of sulfide and hydrosulfide anions in water. Chemical Communications 54, 5859-5862 (2018).
15. Fargher, H. A. et al. Tuning Supramolecular Selectivity for Hydrosulfide: Linear 15. Free Energy Relationships Reveal Preferential C—H Hydrogen Bond Interactions. Journal of the American Chemical Society 142, 8243-8251 (2020).
16. Metrangolo, P., Meyer, F., Pilati, T., Resnati, G. & Terraneo, G. Halogen bonding in supramolecular chemistry. Angewandte Chemie—International Edition vol. 47 6114-6127 (2008).
17. Cavallo, G. et al. The halogen bond. Chemical Reviews vol. 116 2478-2601 (2016).
18. Brown, A. & Beer, P. D. Halogen bonding anion recognition. Chemical Communications 52, 8645-8658 (2016).
19. Lim, J. Y. C. & Beer, P. D. A pyrrole-containing cleft-type halogen bonding receptor for oxoanion recognition and sensing in aqueous solvent media. New Journal of Chemistry 42, 10472-10475 (2018).
20. Langton, M. J., Robinson, S. W., Marques, I., Félix, V. & Beer, P. D. Halogen bonding in water results in enhanced anion recognition in acyclic and rotaxane hosts. Nature Chemistry 6, 1039-1043 (2014).
21. Li, X., Lim, J. Y. C. & Beer, P. D. Acid-Regulated Switching of Metal Cation and Anion Guest Binding in Halogen-Bonding Rotaxanes. Chemistry—A European Journal 24, 17788-17795 (2018).
22. Borissov, A. et al. Anion Recognition in Water by Charge-Neutral Halogen and Chalcogen Bonding Foldamer Receptors. Journal of the American Chemical Society 141, 4119-4129 (2019).
23. Brynn Hibbert, D. & Thordarson, P. The death of the Job plot, transparency, open science and online tools, uncertainty estimation methods and other developments in supramolecular chemistry data analysis. Chemical Communications 52, 12792-12805 (2016).
24. http://supramolecular.org/.
25. X. Wang, L. A. Q. T. and K. C. —RSC Adv. 9, 33578-33583 (2019).
26. F. Weinhold, C. R. L. Valency and Bonding: A Natural Bond Orbital Donor-Acceptor Perspective. Cambridge University Press (2005).
27. R. F. W. Bader. Atoms in Molecules: A Quantum Theory. Oxford University Press (1991).
28. E. V Bartashevich, V. G. T. Russ. Chem. Rev. Russ. Chem. Rev 83, 1181-1203 (2014).
29. J. Wang, R. M. W. J. W. C. P. A. K. and D. A. C. Comput. Chem. Comput. Chem 25, 1157-1174 (2004).
30. J. Wang, R. M. W. J. W. C. P. A. K. and D. A. C. J. Comput. Chem. J. Comput. Chem 26, 114-undefined (2005).
31. C. I. Bayly, P. C. W. D. C. and P. A. K. J. Phys. Chem. J. Phys. Chem 97, 10269-10280 (1993).
32. P. Li, L. F. S. and K. M. M. J. Chem. Theory Comput. J. Chem. Theory Comput. 11, 1645-1657 (2015).

1. A compound having a structure according to Formula I, shown below: 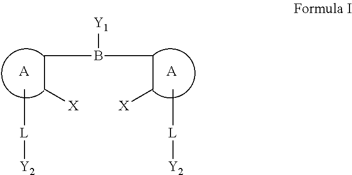
wherein
each X is independently I or Br;
each ring A is independently:
i) an electron deficient 5- or 6-membered nitrogen-containing heteroaryl that is optionally substituted with one or more groups Rₑ, where each Rₑ is independently selected from (1-6C)alkyl, oxygen and aryl;
or
ii) a phenyl that is substituted with one or more groups R_{f}, where each R_{f} is an electron withdrawing group;
each L is independently absent (such that ring A is directly bound to Y₂) or a π-conjugated linker that separates ring A from Y₂ by a distance of 4 atoms or fewer;
Y₁ is absent or is a fluorophore comprising coumarin or BODIPY;
Y₂ is absent or is a fluorophore comprising coumarin or BODIPY;
B is a backbone group of the following structure: 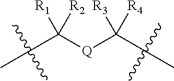
wherein [symbol] denotes the point of attachment to each ring A; and
i) R₁, R₂, R₃ and R₄ are independently selected from hydrogen, halo, hydroxy, (1-4C)alkyl, (2-4C)alkenyl, (2-4C)alkynyl, (1-4C)haloalkyl and (1-4C)alkoxy; and
Q is selected from CRₐR_{b}, N⁺RₐR_{b} and NR_{c}, where Rₐ, R_{b} and Rₑ are each independently hydrogen, (1-6C)alkyl or aryl;
or
ii) R₁ and R₄ are absent, and R₂ and R₃ are linked to one another, such that when taken in combination with the atoms to which they are attached, they form a 5- or 6-membered heteroaryl or an aryl that is optionally substituted with one or more groups Rₐ, where each R_{d} is independently selected from halo, hydroxy, nitro, cyano, (1-6C)alkyl, (2-6C)alkenyl, (2-6C)alkynyl, (1-6C)haloalkyl, (1-6C)alkoxy, —NR_{d1}R_{d2}, —C(O)—R_{d1}, and —C(O)—OR_{d1}, where R_{d1} and R_{d2} are each independently selected from hydrogen and (1-4C)alkyl; and
Q is an atom in the 5- or 6-membered heteroaryl ring or the aryl ring;
with the proviso that Y₁ and Y₂ are not both absent; and
wherein the compound of Formula I is substituted with one or more water solubilising groups, Z.

2. The compound according to claim 1, wherein R₁, R₂, R₃ and R₄ are independently selected from hydrogen, (1-3C)alkyl, (1-3C)haloalkyl and (1-3C)alkoxy; and
Q is selected from N⁺RₐR_{b} and NR_{c}.

3. (canceled)

4. The compound according to claim 1, wherein Rₐ, R_{b} and R_{c} are each independently hydrogen or methyl.

5. The compound according to claim 1, wherein R₁ and R₄ are absent, and R₂ and R₃ are linked to one another, such that when taken in combination with the atoms to which they are attached, they form a 5- or 6-membered heteroaryl or a phenyl that is optionally substituted with one or more groups R_{d}, wherein each R_{d} is independently selected from nitro, (1-3C)alkyl, —C(O)—R_{d1}, and —C(O)—OR_{d1}, where R_{d1} is selected from hydrogen and (1-2C)alkyl; and
Q is an atom in the 5- or 6-membered heteroaryl ring or the aryl ring selected from a carbon atom and a nitrogen atom.

6. (canceled)

7. The compound according to claim 1, wherein B is a backbone group having a structure selected from the following: 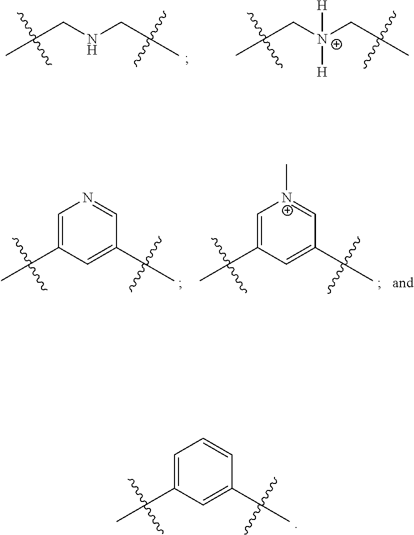
wherein [symbol] denotes the point of attachment to each ring A.

8. The compound according to claim 1, wherein each X is I.

9. The compound according to claim 1, wherein each ring A is independently (i) a triazole or an oxadiazole that is optionally substituted with one or more groups Rₑ, or (ii) a phenyl that is substituted with one or more groups R_{f}.

10. The compound according to claim 1, wherein each Rₑ is independently selected from (1-3C)alkyl, oxygen and phenyl and each R_{f} is independently selected from fluoro, chloro, nitro and cyano.

11. The compound according to claim 1, wherein each L is absent.

12. The compound according to claim 1, wherein Y₁ and Y₂ are not both a fluorophore comprising coumarin or BODIPY.

13. The compound according to claim 1, wherein Y₁ is absent and Y₂ is a fluorophore selected from: 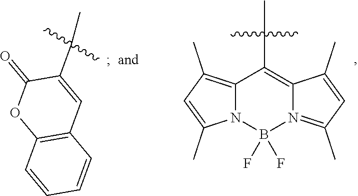
wherein [symbol] denotes the point of attachment to L (or ring A when L is absent).

14. (canceled)

15. The compound according to claim 1, wherein the one or more water solubilising groups, Z, is independently selected from one of the following structures: 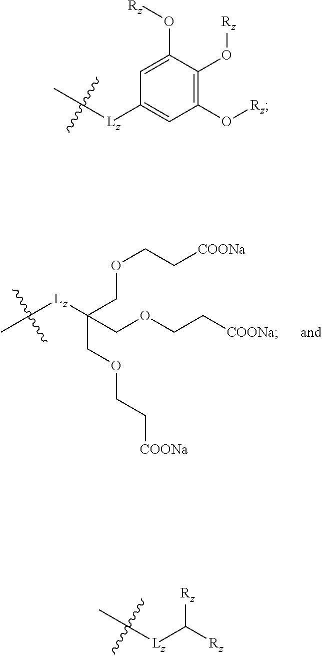
wherein [symbol] denotes the point of attachment of Z within the compound of Formula I (e.g. the point of attachment to Y₂ or B);
L_{Z} is absent (i.e. bond) or is a linking group containing 15 atoms or fewer in total; and
R_{z} is a polyethylene glycol group, each comprising 2-10 (or 2-8 or 3-6) repeating ethylene glycol units.

16. The compound according to claim 1, wherein the compound of formula I has a structure according to Formula II, shown below: 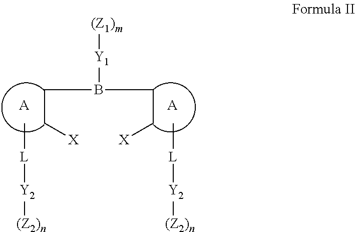
wherein B, A, L, X, Y₁ and Y₂, and any associated sub-groups, have any of the definitions outlined in claim 1;
Z₁ and Z₂ are each independently a water solubilising group having any of the definitions outlined for Z in claim 1;
m is 0, 1 or 2; and
n is 0, 1 or 2;
with the proviso that m and n are not both 0.

17. The compound of claim 16, wherein
m is 0 and n is 1; and
Y₁ is absent and Y₂ is a fluorophore: 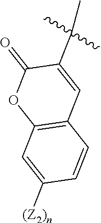
wherein [symbol] denotes the point of attachment to L (or ring A when L is absent).

18. The compound of claim 16, wherein the compound of Formula II has a structure according to Formula IIa-v or IIa-vi shown below: 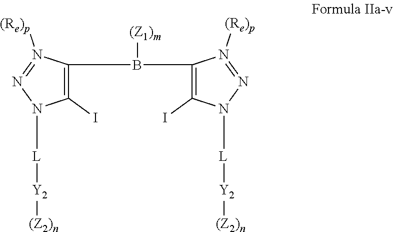 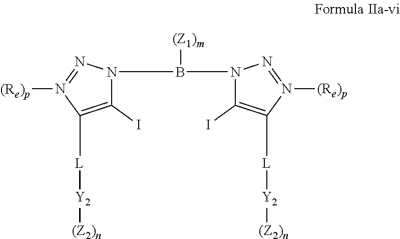
wherein
p is 0 or 1; and
Y₂, m, n, Rₑ, Z₁, Z₂, B and each L, and any associated sub-groups, have any of the definitions outlined in any one of the preceding claims claim 1.

19. The compound according to claim 1, wherein the compound is selected from any one of the following:

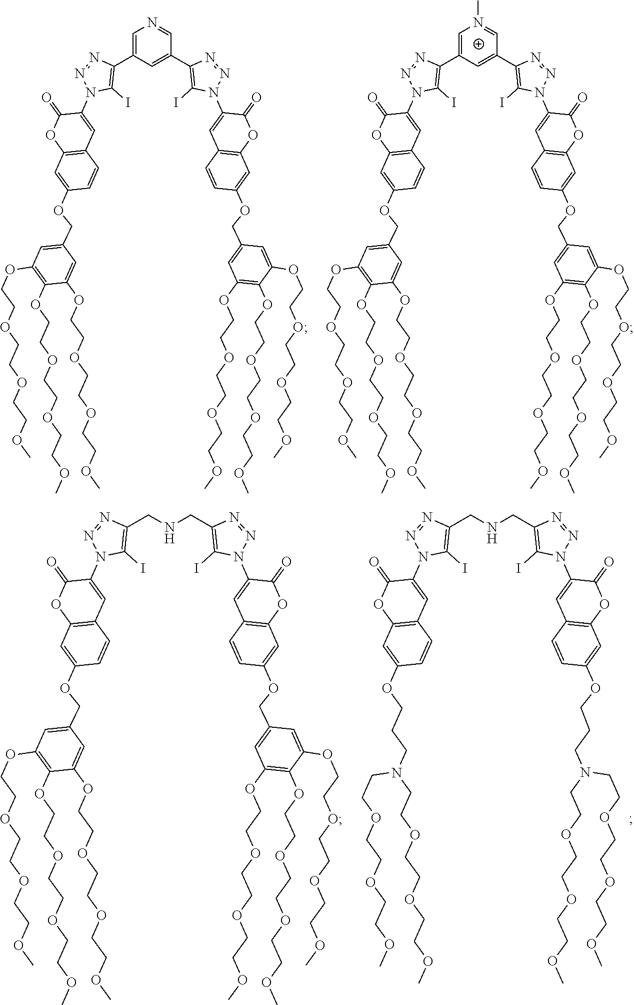

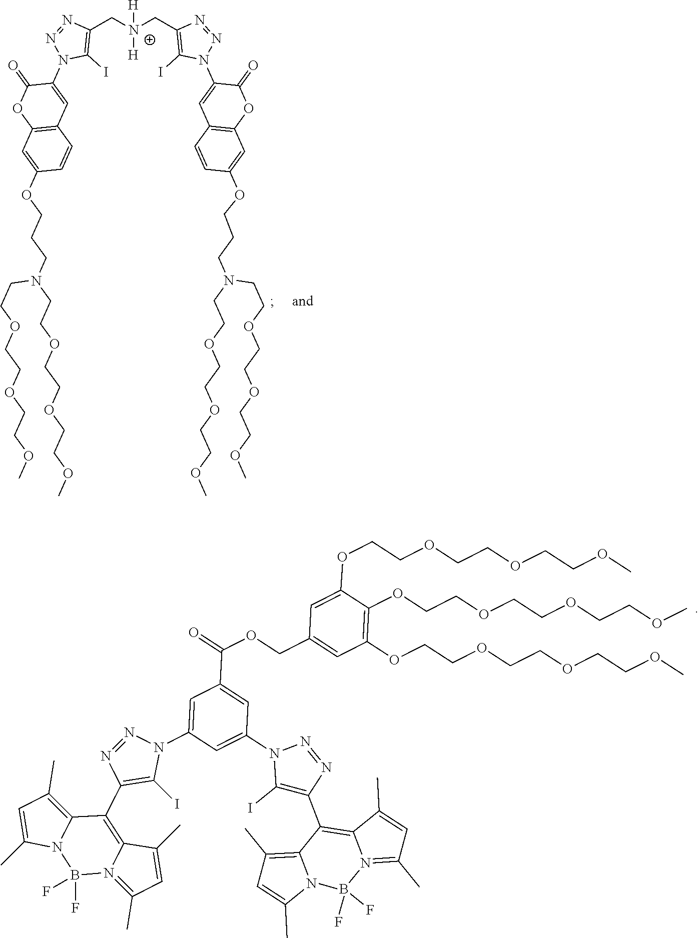

20. The compound according to claim 1, wherein the compound is bound to a solid support.

21. A sensor comprising a compound according to claim 1.

22. (canceled)

23. A method of detecting the presence or absence of sulphide in a solution, the method comprising:
a) contacting a compound according to claim 1 or a sensor according to claim 1 with the solution; and
b) detecting the presence or absence of sulphide in the solution.

24. The use according to claim 23, wherein the solution is: (i) an aqueous solution, or (ii) wastewater.

25. (canceled)
